(19) United States
(12) Patent Application Publication
(10) Pub. No.: US 2021/0213250 A1
(43) Pub. Date: Jul. 15, 2021

(54) CATHETER WITH BRAID AND RADIOPAQUE SECTION

(71) Applicants: Jacob Chmielewski, Clear Lake, MN (US); Karl V. Ganske, Hopkins, MN (US)

(72) Inventors: Jacob Chmielewski, Clear Lake, MN (US); Karl V. Ganske, Hopkins, MN (US)

(21) Appl. No.: 17/145,092

(22) Filed: Jan. 8, 2021

Related U.S. Application Data
(60) Provisional application No. 62/959,645, filed on Jan. 10, 2020.

(51) Int. Cl. A61M 25/01; A61M 25/00

(52) U.S. Cl. CPC A61M 25/0108; A61M 2025/0059; A61M 25/0045; A61M 25/0053

(57) ABSTRACT

In an example, a catheter assembly includes a catheter body extending from a catheter proximal portion to a catheter distal portion having a distal tip. a coil proximate to the catheter distal portion, the coil having a first section and a second section, wherein the first section has a first imaging characteristic. The second section is swaged, and the second section of the coil has a second imaging characteristic. The second imaging characteristic differs from the first imaging characteristic, and the different first and second imaging characteristics are configured to contrast and emphasize visibility of the distal tip relative to the remainder of the catheter body. In another example, a second braided layer of the catheter body is configured to constrain and brace a first braided layer.

### CLAIM OF PRIORITY

This patent application claims the benefit of priority of Chmielewski U.S. Provisional Patent Application Ser. No. 62/959,645, entitled “CATHETER WITH BRAID AND RADIOPAQUE SECTION,” filed on Jan. 10, 2020 (Attorney Docket No. 3028.053PRV), which is hereby incorporated by reference herein in its entirety.

### TECHNICAL FIELD

This document pertains generally, but not by way of limitation, to catheters including instruments used in diagnostic or therapeutic procedures.

### BACKGROUND

Introducer sheaths, guide catheters and the like are used for diagnostic and therapeutic procedures. Sheaths and guide catheters are used to guide other instruments including catheters into and through the vasculature to one or more locations of interest. Catheters are delivered through one or more of a sheath or guide catheter, and are optionally navigated with stylets, guidewires or the like through vasculature beyond the distal ends of the sheath or guide catheter. In some examples, the catheter is axially loaded (e.g., pushed). An imaging device (e.g., ultrasound prove, X-ray, CAT scan, or the like) is optionally utilized to determine a location of the catheter when the catheter is located in anatomy of a patient (e.g., a blood vessel, or the like).

### SUMMARY

In an approach, a radiographic marker is included proximate to a distal end of a catheter, for instance located at a distal tip of the catheter. Orientation of the catheter can be difficult to assess, for instance if the radiographic marker is a ring at a portion of the distal tip. Accordingly, the radiographic marker can be difficult to observe through imaging, such as x-ray imaging. For instance, in some approaches the relatively small radiographic marker is difficult to discern, and other portions of the catheter are also difficult to discern. The present inventors have recognized, among other things, that a problem to be solved can include precisely locating one or more portions of a catheter when the catheter is located in anatomy of a patient. The present subject matter can help provide a solution to this problem, such as by providing a catheter assembly including a catheter body extending from a catheter proximal portion to a catheter distal portion having a distal tip. In some examples, the catheter body includes a coil proximate to the catheter distal portion. The coil optionally has a first section and a second section. These sections facilitate the identification of the distal tip, and at the same time provide an indication of the orientation of the distal portion of the catheter including the distal tip. In an example, the first section of the coil extends toward the distal tip. The first section optionally has a first imaging characteristic (e.g., a first radiodensity, or the like).

In an example, the second section of the coil is swaged, and the second section extends from the first section to the distal tip. The second section of the coil optionally has a second imaging characteristic (e.g., a second radiodensity). In some examples, the second imaging characteristic differs from the first imaging characteristic, and is accordingly observably distinct from the first section with one or more imaging techniques, such as fluoroscopy. The different first and second imaging characteristics of the sections of the coil are configured to contrast and emphasize visibility of the distal tip relative to the remainder of the catheter body of the catheter assembly. Accordingly, the catheter assembly facilitates locating the distal tip when the catheter is located in the anatomy of a patient, and further provides an indication of the orientation of the distal tip by way of variation in the observable first and second sections.

The present inventors have recognized, among other things, that a problem to be solved can include providing a flexible distal tip, for instance while contrasting and emphasizing visibility of the distal tip relative to the remainder of the catheter body. In some examples, a marker band is coupled to the catheter body. The marker band provides contrast and emphasizes visibility of the distal tip. However, in some examples the marker band is rigid and inhibits the flexibility of the distal tip (e.g., a portion of the catheter body proximate the catheter distal portion). Flexibility of the catheter body improves the ability of the catheter assembly to navigate circuitous anatomy of a patient (e.g., one or more blood vessels, arteries, or the like).

The present subject matter can help provide a solution to this problem, such as by improving the flexibility of the distal tip while at the same time providing enhanced visibility of the distal tip and orientation of the distal tip. In an example, the second section of the coil extends to the distal tip, and the second section of the coil is included in the distal tip. As described herein, the second section of the coil contrasts and emphasizes visibility of the distal tip relative to the remainder of the catheter body. Accordingly, the catheter assembly provides flexibility to the distal tip while providing contrast and emphasizing visibility of the distal tip relative to the remainder of the catheter body.

The present inventors have recognized, among other things, that a problem to be solved can include improving the pushability of the catheter assembly, for instance by reducing kinking of the catheter body when the axial force is applied to the catheter assembly. In an example, an axial force is applied to the catheter assembly to translate the catheter body distally relative to the anatomy (e.g., vasculature, or the like) of a patient. The catheter body is flexible to facilitate navigating the anatomy. In some examples, the catheter body kinks (e.g., bends, creases, folds, or the like) due to the flexibility of the catheter body.

The present subject matter can help provide a solution to this problem, such as by providing a first braided layer within the catheter body. A second braided layer is optionally coupled along an exterior of the first braided layer. The first and second braided layers include first and second, respective, filar arrays. In some examples, the first and second braided layers expand to an expanded configuration with the application of axial force along the catheter length axis. In the expanded configuration the first braided layer optionally expands at a first rate. The second braided layer optionally expands at a second rate, and in some examples the second rate of expansion is different (e.g., less or greater) than the first rate of expansion. The second braided layer optionally constrains and braces the first braided layer, for instance because the second braided layer expands at a lesser rate than the first braided layer. The first braided layer is thereby constrained and supported (e.g., braced) by the more slowly expanding second braided layer. The constraining and bracing of the first braided layer by the second braided layer improves the pushability of the catheter assembly and minimizes buckling or kinking, for instance by increasing an amount of axial force needed to kink the catheter body.

This overview is intended to provide an overview of some of the subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a front view of a catheter, according to an embodiment.

FIG. 2 is a back view of a catheter, according to an embodiment.

FIG. 3 is a cross-sectional view of a catheter, according to an embodiment.

FIG. 4 is a cross-sectional view of a portion of a catheter, according to an embodiment.

FIG. 5 is a cross-sectional view of a portion of a catheter, according to an embodiment.

FIG. 6 is a cross-sectional view of a portion of a catheter, according to an embodiment.

FIG. 7 is a cross-sectional view of a portion of a catheter, according to an embodiment.

FIG. 8 is a cross-sectional view of a portion of a catheter, according to an embodiment.

FIG. 9 is a cross-sectional view of a portion of a catheter, according to an embodiment.

FIG. 10 is a front view of a catheter, according to an embodiment.

FIG. 11 is a composite cross sectional view of one example catheter taken along a longitudinal axis (right view) and an orthogonal axis (left view).

FIG. 12 is a composite sectional view of another example catheter having a partial section along a longitudinal axis (right view) and a cross section along an orthogonal axis (left view).

FIG. 13 is a perspective view of one example of a catheter assembly including a graduated strain relief fitting.

FIG. 14 is a side view of another example of a catheter assembly in a deflected configuration including a stress riser.

FIG. 15 is a side view of the graduated strain relief fitting of FIG. 1.

FIG. 16A is a cross sectional view of the graduated strain relief fitting of FIG. 1.

FIG. 16B is a cross sectional view of another example of a graduated strain relief fitting including a plurality of fitting materials.

FIG. 17 is a side view of the catheter assembly of FIG. 1 in a deflected configuration with the graduated strain relief fitting having a complementary profile to the deflected catheter shaft.

FIG. 18A is a detailed side view of another example of a graduated strain relief fitting.

FIG. 18B is a detailed side view of an additional example of a graduated strain relief fitting.

FIG. 18C is a detailed side view of a supplemental example of a graduated strain relief fitting.

FIG. 18D is a detailed side view of yet another example of a graduated strain relief fitting.

FIG. 19 is a cross-sectional view of the catheter 100 of FIG. 1 at the line 19-19.

FIG. 20 is a detailed cross-sectional view of the catheter assembly of FIG. 19 at the circle detail 20.

FIG. 21 is a side view of the catheter body.

FIG. 22 is a detailed side view of the catheter body of FIG. 21 at the box detail 22 in FIG. 21.

FIG. 23 is a cross-sectional view of the of the catheter body of FIG. 22 at the line 23-23.

FIG. 24 is a detailed cross-sectional view of the catheter body of FIG. 23 at the box detail 24.

FIG. 25 is a schematic view of another example of the catheter including a first braided layer.

FIG. 26 is a schematic view of another example of the catheter including a second braided layer.

FIG. 27 is a schematic view of the catheter having a braided assembly including the first braided layer and the second braided layer.

FIG. 28 is a schematic view of the first braided layer and the second braided layer in an initial configuration.

FIG. 29 is a schematic view of the first braided layer and the second braided layer in an expanded configuration.

### DETAILED DESCRIPTION

In an example, a catheter includes a catheter body having a coil. The coil is proximate to a distal portion of the catheter body. The coil includes a first section having a first imaging characteristic (e.g., radiopacity, radiodensity, radiopaqueness, radiolucentness, ultrasound opacity or the like). The coil includes a second section having a second imaging characteristic. The second imaging characteristic differs from the first imaging characteristic, and the different first and second imaging characteristics contrast and emphasize visibility of the distal tip relative to the remainder of the catheter body. Accordingly, the flexibility of the distal tip is enhanced with the coil, while the coil provides enhanced visibility of the distal tip and orientation of the distal tip.

In another example, the catheter body includes a braid assembly (e.g., an assembly including the first braided layer 2500 and the second braided layer 2600) that improves the pushability of the catheter assembly and minimizes buckling or kinking, for instance by increasing an amount of axial force needed to kink the catheter body. For instance, the catheter body includes a first braided layer and a second braided layer coupled along an exterior of the first braided layer. The second braided layer optionally constrains and braces the first braided layer, for instance because the second braided layer expands at a lesser rate than the first braided layer. The constraining and bracing of the first braided layer by the second braided layer, for instance with perimeter support provided by the second braided layer, improves the pushability of the catheter assembly and minimizes buckling or kinking, for instance by increasing an amount of force needed to kink the catheter body.

The catheter assemblies described herein include features usable alone, or in combination with other features described herein. For instance, the catheter assembly 100 described herein optionally includes (but is not limited to) one or more of the braid assembly 112, braid assembly 608, braid assembly 808, braid assembly 814, braid assembly 1100, braid assembly 1200, strain relief fitting 110, graduated strain relief fitting 1300, braid assembly 2700, or the like.

Additionally, the catheter examples as described herein can solve the problems associated with current catheter technology by providing novel designs, construction and materials. The catheters, described herein, are optionally used in interventional procedures including access to one or more vessels or passages (e.g., arteries, veins, vessels, body passages or cavities and the like). Further, the catheters described herein facilitate enhanced deflection, torqueability and other mechanical characteristics of the catheter during navigation while at the same time minimizing kinking. For instance, where significant arterial tortuosity is encountered such with a radial artery access or a femoral approach on an obese patient, the catheters described herein are configured for navigation through such vessels. The catheters described herein include, but are not limited to, introducer sheaths, guide catheters, delivery catheters, or other typically tubular devices used in diagnostic or therapeutic procedures (e.g., including instruments, fluid delivery passages, balloons or the like).

In various embodiments, the catheters include a composite built tube fabricated using a wound metal inner layer (e.g., a braid assembly, coil or the like) and jacketed with layers of polymer inside and out, for instance an inner liner and outer sleeve, respectively. The metallic inner layer is optionally constructed with a multi-filar (6-30 filars) helically wound braid structure. In some embodiments, the filars are swaged, such that one or more of the filars is partially flat or ovular (e.g., including rectangular) in cross-section to achieve a tight wire matrix. In other examples, the braid assembly is made with one or more non-swaged, round, square or rectangular filars (optionally in combination with other filars having the swaged configuration). As described herein, the braid assembly includes filar arrays, for instance first and second filar arrays that are helically wound and interlaced.

In various embodiments, the wall thickness of the braid assembly ranges from about 0.0005 to 0.020 inches thick. The braid assembly improves the mechanical integrity of the catheter, such as compared to current guide catheters with respect to kinking, buckling, flexibility, radial strength, and maintaining circularity of the catheter lumen cross-section. This improvement is achieved in one example by varying number of filars in each of the filar arrays (e.g., in various ratios including but not limited to, 15:1, 14:2, 13:3, 12:4, 11:5, 10:6, 9:7, 8:8, 7:9, 6:10, 5:11, 4:12, 3:13, 2:14, 1:15 with a total filar count of 16 filars).

In another example, the improved mechanical characteristic (or characteristics) is achieved by varying dimensions (e.g., dimensions in cross section) of one or more of the filars in one or more of the arrays. For instance, in arrays with the ratio 14:2 the first filars (14 filars) include first filar dimensions in the cross section such as one or more of diameter, thickness or width less than corresponding dimensions of the second filars (2 filars). Stated another way, the second filars are fewer in number and are larger in at least one cross sectional dimension relative to the first filars. As described herein, the second filars structurally support the more narrow first filars in the manner of a braided brace, and thereby behave as a frame, skeleton, cage or the like that maintains the first filars in a desired configuration (e.g., without kinking or buckling during deflection). Optionally the second filars include one or more filars, such as coils, interlaced with the first filars. The coils act as a braided brace for the first filars similar to the second filars previously described. The first filars (and the second filars including the braided brace) provide improved torqueability to the catheter, while the braided brace structurally supports the braid assembly and provides at least enhanced kink resistance. The inclusion of a braided brace incorporates the profile of the brace into the braid assembly and thereby avoids coupling additional support structures over or beneath the braid assembly with attendant consumption of space (or enlargement of the catheter) avoided.

In still another example, the catheters described herein include a coil wound along the braid assembly, for instance in a guide recess provided by one of the filar arrays. The coil enhances the mechanical characteristics of the catheters. Optionally, the coil extends helically along one or more of the filars including for example the braided brace. The coil and the braided brace cooperate to capture and hold the first filars (described above) in place within the catheter. Further, infiltration of the outer sleeve (e.g., a reflowed or shrunk sleeve) into the braid assembly and the optional coils fixes each of the braided brace and the coil (optional) in place. The outer sleeve and the braided brace capture and hold the first filars in place and minimize (e.g., eliminate or decrease) kinking of the catheters. Where the coil is included along the braid assembly, the coil and the braided brace are captured within the outer sleeve (e.g., reflowed) and clamp the first filars therebetween. Kinking, buckling or the like of the first filars is thereby resisted by one or more of the braided brace or the coil in combination with the outer sleeve.

In at least some examples, the catheter of this disclosure also comprises an outer sleeve, such as an outer polymer layer and an inner liner, such as an inner polymer layer. In an embodiment, the outer polymer layer and the inner polymer layer include one or more polymers, such as PTFE, Pebax, or Polyurethane. The polymer layers are attached to the braid assembly by thermal polymer heat-shrinking or reflow. The wall thickness of the polymer layers ranges from 1.0 to 3.0 thousandths of an inch for each layer.

In various embodiments, the catheters include a pre-shaped curve, such as a curved distal end region. The catheter attains the pre-shaped curve configuration by, for instance, heat-setting the metal portion of the catheter where the curved configuration is specified. The curve retains its shape in body temperature and over time does not substantially soften (e.g., to unintentionally change shape). The guide catheter optionally includes a soft (low durometer) polymer distal tip, various distal curve shapes, a radiopaque distal marker band, a proximal luer adapter or the like.

The catheters described herein range in size from 3F to 34F and in lengths from at least 15 cm or more to 205 cm or less. As previously described, the features, elements and functions described herein as well as their equivalents are used in a variety of catheters including, but not limited to, introducer sheaths, guide catheters, catheters including one or more of instruments or delivery lumens, or the like. That is to say, the enhancements to each of torqueability, pushability, flexibility, kink-resistance or the like are readily applied to various catheter styles and types.

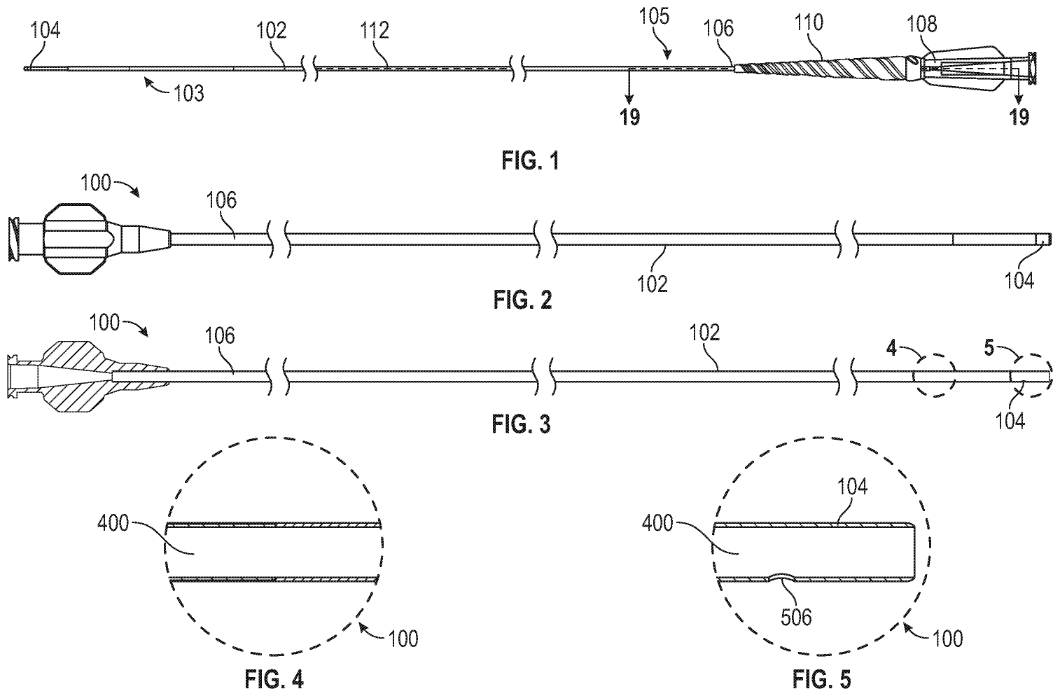

In reference now to the Figures, FIG. 1 shows a front view of a guide catheter 100, according to an embodiment. FIG. 2 shows a back view of the guide catheter 100. FIG. 3 shows a cross-sectional view of the guide catheter 100 shown in FIGS. 1 and 2. In an embodiment, the guide catheter 100 can be configured for introducing interventional catheters into the vasculature of a patient.

In an embodiment, the catheter 100 includes a catheter body 102 (e.g., a main tubular shaft with an optional lumen) with a distal portion 103 (including a distal tip 104) and a proximal portion 105 (including a proximal end 106). The distal tip 104 is on the opposed end of the catheter body 102 from the proximal end 106. The distal tip 104 includes at least one layer of polymer. In another example, the distal tip 104 includes at least two layers of polymer. Optionally, the distal tip 104 includes an inner layer and an outer layer. In one example, the inner layer of the distal tip 104 includes PTFE. In another example, the outer layer of the distal tip 104 includes Pebax. In an embodiment, the distal tip 104 has a length of at least 0.05 inches. In another embodiment, the distal tip 104 has a length of at least 0.02 inches long. In yet another embodiment, the distal tip 104 is 0.2 inches long or shorter. In a further embodiment, the distal tip 104 can be is 0.5 inches long or less.

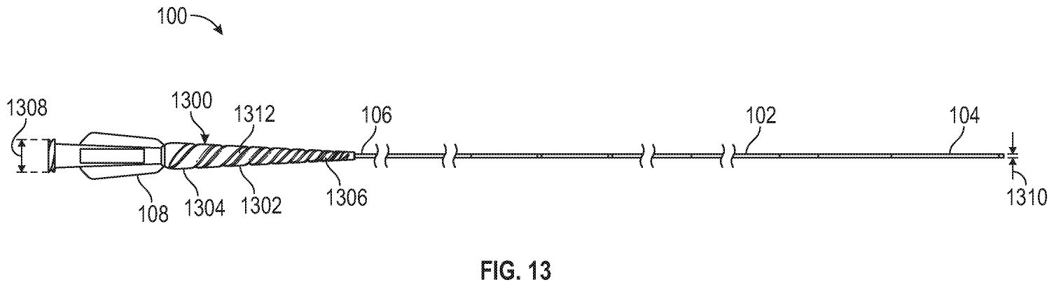

In an example, the catheter assembly 100 includes a hub 108 coupled with the catheter body 102. For instance, the hub 108 is included in a proximal portion of the catheter assembly. In another example, the proximal end 106 of the catheter body 102 is coupled with the hub 108. In yet another example, the hub 108 includes a strain relief fitting 110. For instance, in some examples, the catheter assembly 100 includes the graduated strain relief fitting 1300 (shown in FIG. 13) configured to support the catheter body 102 and minimize kinking of the body proximate to the fitting 1300. In another example, the catheter body 102 includes a braid assembly 112. The braid assembly 112 enhances performance of the catheter assembly 100, for example by reducing kinking (or buckling) of the catheter body 102. In yet another example, the catheter assembly 100 includes the braid assembly 2700 having a first braided layer (e.g., second braided layer 2600, shown in FIG. 27) constrains and braces a second braided layer (e.g., first braided layer 2500, shown in FIG. 27) to reduce one or more of kinking or buckling of the catheter body 102.

In various embodiments, the catheter body (e.g., including the catheter body 102) includes a main inner structural layer, for instance one or more of the braid assembly, discrete coil or combinations of the same as described herein. The main inner structural layer includes a helically interlaced braid assembly extending between the proximal end portion and the distal end portion (e.g., along the entire length or a portion of the length of the catheter body). In various embodiments, the braid assembly covers at least a portion of the inner liner of the catheter. The outer sleeve, for instance a shrink tube, reflowed polymer or the like surrounds the braid assembly and in at least some examples infiltrates interstitial spaces of the braid assembly (e.g., between filars, coils, opposed helixes of the braid or the like).

As described herein, the catheter body including the catheter body 102 includes an outer layer (e.g., a jacket, such as an outer sleeve). The outer layer optionally includes a polymer. The outer layer surrounds the braid assembly (e.g., jackets, coats, covers or the like). The outer layer is fixed (e.g., fixedly coupled) to the braid assembly and optionally the inner liner through one or more of shrinking of the outer layer (shrink tubing) or infiltration of the braid assembly and optionally contacting the inner liner (by reflowing).

In various embodiments, the inner liner of the catheter (e.g., the catheter body including the catheter body 102) includes a polymer. The inner layer (e.g., the inner liner) extends along and couples with an inner surface of the braid assembly (e.g., jackets, coat or covers or the like). The inner layer is coupled (e.g., fixedly coupled) to the main inner structural layer through one or more of compression of the braid assembly onto the liner (e.g., with an outer sleeve including a shrink tube), compression achieved during braiding of the braid assembly onto the liner, infiltration of the braid assembly by a reflowed outer sleeve including contact and coupling of the reflowed polymer with the inner liner.

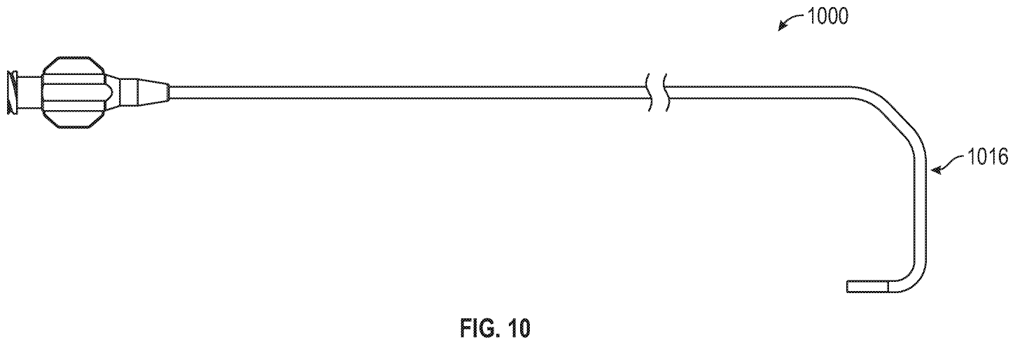

The catheter body 102 optionally includes a curve, for instance at or near the distal end portion including the distal tip 104 (shown in FIG. 10). The curve shape is configured for anatomical conformance. The shape is optionally formed and heat processed into the catheter body 102, such as in the braid assembly or another metal portion. In one example, the braid assembly terminates distally prior to the curve of the distal end portion.

In various embodiments, the braid assembly is laminated between the inner layer (inner liner) and the outer layer (outer sleeve), such that the lamination does not fuse the outer sleeve and the inner liner together.

In an embodiment, the catheter is at least 60 cm long and not longer than 200 cm. In another embodiment, the catheter body 102 is at least 10 cm long and not longer than 300 cm. In still another embodiment, the catheter body 102 is at least 30 cm long and not longer than 250 cm. In a further embodiment, the catheter body 102 is at least 50 cm long and not longer than 225 cm.

In an embodiment, the catheter body 102 includes an outer diameter of at least 0.060 inches and not more than 0.115 inches (e.g., the outer diameter of the outer sleeve when coupled with the remainder of the catheter body). In another embodiment, the catheter body 102 includes an outer diameter of at least 0.060 inches. In a further embodiment, the catheter body 102 includes an outer diameter of at least 0.040 inches. In yet another embodiment, the catheter body 102 includes an outer diameter of at least 0.050 inches. In further embodiments, the catheter body 102 includes an outer diameter of at least 0.070 inches, at least 0.080 inches or the like. In still another embodiment, the catheter body 102 has an outer diameter of no greater than 0.115 inches. In further examples, the catheter body 102 has an outer diameter including, but not limited to, no greater than 0.095 inches, no greater than 0.105 inches, no greater than 0.125 inches, no greater than 0.135 inches.

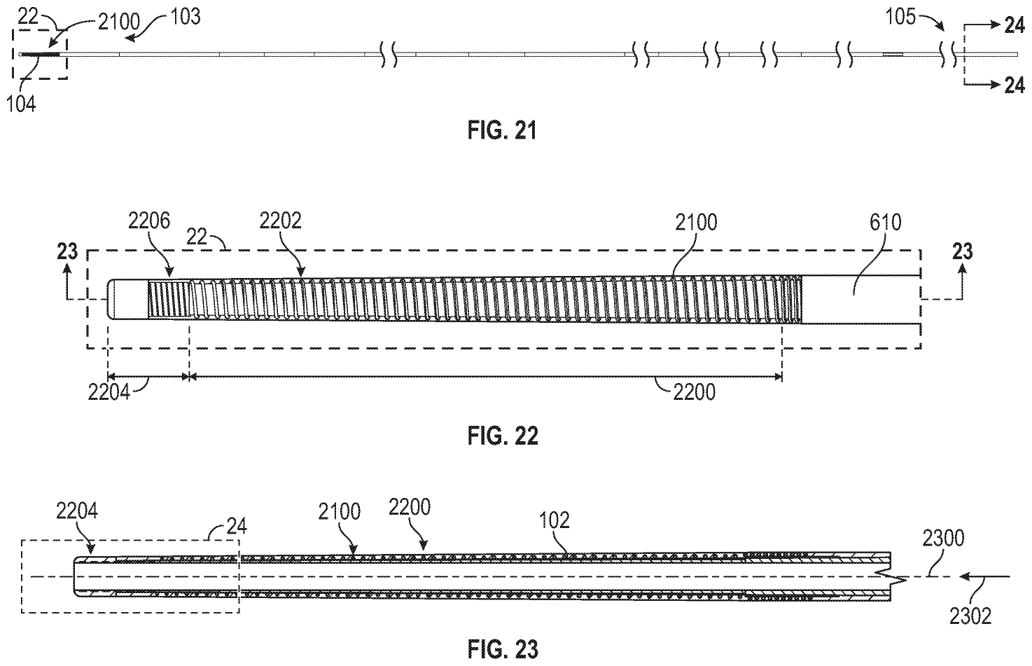

FIG. 4 and FIG. 5 show cross-section views of portions of a catheter 100, according to various embodiments. In the example shown, the catheter 100 includes an inner lumen 400. In other examples, the catheter is without an open inner lumen. FIG. 5 shows a cross-section of a portion of an example distal tip 104. As seen in FIG. 5, the guide catheter 100 in an example includes one or more apertures 506. In various embodiments, the catheter body including the catheter body 102 includes an aperture 506 extending from the interior of the catheter 100 to the exterior of the catheter. In an embodiment, the distal tip 104 includes the aperture 506. As further described herein, the distal tip 104 includes one or more radio-opaque features, including, but not limited to the coil 2100 (shown in FIG. 21) having first and second imaging characteristics 2202, 2206.

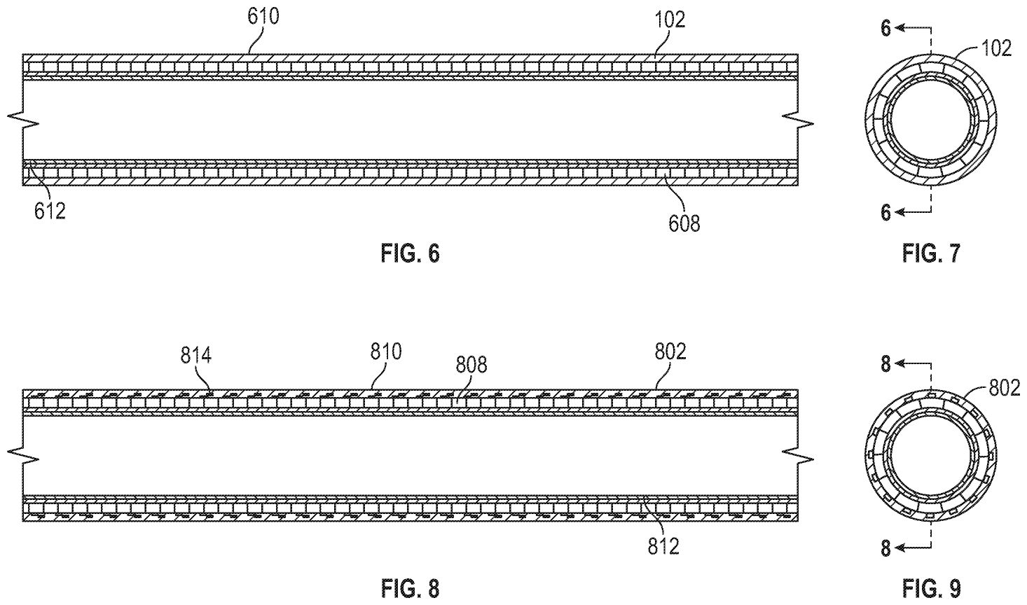

FIG. 6 shows a cross-sectional view of a portion of the catheter body 102 along the longitudinal axis of the catheter 100, according to an embodiment. FIG. 7 shows a cross-sectional view from the end of the catheter body 102 (e.g., orthogonal to the longitudinal axis of the catheter 100). In an embodiment, the catheter body including the catheter body 102 includes the braid assembly 608. The braid assembly 608 includes two or filar arrays interlaced in opposed (left and right) directions around the catheter body. As described herein, each of the filar arrays includes one or more filars including but not limited to flat or ovular (e.g., swaged) filars, coils (circular filars) or the like. In various embodiments, the braid assembly includes one or more filars constructed with, but not limited to, metal (stainless steel, Nitinol or the like), polymers, composites or combinations of filars constructed with two or more of the materials described herein. In other examples described herein, the braid assembly includes filar arrays provided at one or more different orientations. For instance, as shown in FIGS. 25-27, the braid assembly 2700 includes a first braided layer having a first braid profile angled with respect to a second braid profile of a second braided layer. The angle between the first braid profile and the second braid profile minimizes kinking (or buckling) of the catheter shaft 102, for instance to vary rates of expansion between the first braided layer and the second braided layer. Accordingly, in some examples the second braided layer constrains and braces the first braided layer due to variations in expansion between the braided layers.

In various embodiments, the braid assembly 608 filars are swaged. In various embodiments, the braid assembly component filars includes include between at least 2 and 30 filars having a picks per inch (inverse of pitch) of between 30 and 180. In various embodiments, the braid assembly includes at least 4 filars and not more than 24 filars. In other embodiments, the braid assembly includes at least 8 filars and not more than 16 filars. In various embodiments, the metallic filars of the braid assembly 608 include cross sectional shapes including, but not limited to, rectangular cross-sections, circular cross-sections, ovular cross-sections, elliptical cross-sections (another example of an oval) or the like. In various embodiments, the braid assembly 608 filars are coated, for instance with PTFE, prior to braiding into the interlaced configuration of the braid.

In an embodiment, the braid assembly 608 includes welded terminations. In an embodiment, the braid assembly 608 includes a distal end having a gold coating. In various embodiments, the gold coating ranges from about 0.5 mm to 2 mm thick. In other embodiments, the gold coating ranges from about 0.4 mm to 2.5 mm thick. In still other embodiments, the gold coating ranges from 0.25 mm to 3 mm thick.

In an embodiment, the braid assembly 608 includes thickness (e.g., from the braid assembly exterior to the braid assembly interior) that ranges from about 0.0015 inches to 0.010 inches. In another embodiment, the braid assembly 608 includes a thickness of at least 0.0010 inches. In still another embodiment, the braid assembly 608 includes a thickness of at least 0.0005 inches. In other embodiments, the main inner braid assembly 608 includes a thickness of no greater than 0.015 inches. In still other embodiments, the braid assembly 608 includes a thickness of no greater than 0.020 inches.

As described herein, the catheter body including the catheter body 102 includes an outer layer 610, such as an outer sleeve. The outer layer 610 includes a polymer in at least one example. The outer layer 610 extends around (e.g., jackets, covers, coats or the like) at least a portion of the braid assembly 608. In an embodiment, the outer layer 610 is at least about 0.001 inches thick and not more than about 0.005 inches thick. In another embodiment, the outer layer 610 is least about 0.0007 inches thick. In still another embodiment, the outer layer 610 is at least about 0.0005 inches thick. In yet another embodiment, the outer layer 610 is no more than about 0.007 inches thick. In further embodiments, the outer layer is no more than about 0.01 inches thick.

Optionally, the outer layer 610 includes one or more of polymers including, but not limited to, Pebax, PTFE, shrink tubing or the like. In another example, the outer layer 610 includes nylon. In an embodiment, the outer layer 610 is coated with a hydrophilic polymer. In another example, the outer layer 610 includes at least two layers. Optionally, each of the two layers includes Pebax or one or more of the polymers described herein. In another example, the outer layer 610 is heat shrinkable to snugly couple form the outer layer 610 onto the braid assembly 608. In yet another example, the outer layer 610 includes a reflowable polymer that is heated and reflows around the braid assembly 608. Optionally, the reflowed outer layer 610 infiltrates and captures one or more of the filars (including the braided brace) within the outer layer 610. As described herein, the outer layer 610 in cooperation with the braided brace (e.g., a coil, filars as described herein or the like) and an optional discrete coil maintain the braid assembly 608 in a specified (unkinked) configuration even with significant deflection of the catheter 100 (relative to a braid assembly without the structural support described herein).

In an embodiment, the catheter body including the catheter body 102 includes an inner liner, such as an inner layer 612. The inner layer 612 includes a polymer including, but not limited to, a lubricious polymer such as PTFE (e.g., to provide strength and facilitate passage of instruments through an optional center lumen as shown in FIGS. 6 and 7). The inner layer 612 extends along at least an interior portion of the braid assembly 608.

In an embodiment, the inner layer 612 is at least about 0.001 inches and not more than about 0.005 inches thick. In another embodiment, the inner layer 612 is at least about 0.0007 inches thick. In yet another embodiment, the inner layer 612 is at least about 0.0005 inches thick. In another example, the inner layer 612 is no more than about 0.007 inches thick. In still another example, the inner layer is no more than about 0.01 inches thick. Optionally, the inner layer 612 includes one or more polymers including, but not limited to, PTFE (described above), nylon, and coated polymers (e.g., coated with a hydrophilic polymer).

In an embodiment, the outer layer 610 (outer sleeve) and the inner layer 612 (inner liner) are fused together, for instance through the braid assembly 608 (shown in FIGS. 6 and 8). FIG. 8 shows a cross-section view of a portion of a main tubular shaft 802 (a portion of the catheter body), according to an embodiment. FIG. 9 shows a cross-section view from the end of the main tubular shaft 802. In various embodiments, the main tubular shaft 802 include a braid assembly 814. The braid assembly 814 is disposed between an optional inner braid assembly 808 (or other structural support layer, such as a coil) and the outer layer 810. In another example, the braid assembly 814 is between the outer sleeve (e.g., outer layer 810) and the inner liner (e.g., inner layer 812). In yet another example, the braid assembly 814 is disposed within a portion of the outer layer 810. The braid assembly 814 covers at least a portion of the optional inner braid assembly 808 in another example.

In an embodiment, the filars of the braid assembly 814 includes metal or a polymer including, but not limited to, stainless steel, Nitinol or the like. In one embodiment, the braid assembly 814 is at least about 0.0005 inches thick and not more than about 0.010 inches thick. In another embodiment, the braid assembly 814 is at least about 0.005 inches thick and not more than about 0.010 inches thick. In still another embodiment, the braid assembly 814 is at least about 0.0004 inches thick. In a further embodiment, the braid assembly 814 is at least about 0.0003 inches thick. In other embodiments, the braid assembly 814 no more than about 0.015 inches thick, no more than about 0.020 inches thick or the like.

FIG. 10 shows a front view of an example guide catheter 1000 according to an embodiment. The guide catheter 1000 includes a distal end curve 1016. The distal end curve 1016 is, in one example, configured for anatomical conformance. As described herein, the distal end curve 1016 is heat processed and formed of a formable portion of the catheter 1000, for instance in regions including one or more of the braid assemblies described herein, a separate metal feature or the like. The distal end curve 1016 retains its shape in body temperature and over time does not substantially soften and unspecified shape changes of the curve are thereby prevented.

The catheters described herein include a catheter body including a braid assembly having at least first and second interlaced filar arrays, with each of the filar arrays including one or more respective first and second filars extending in opposed helixes. The braid assembly is between an inner liner and an outer sleeve. In at least one example, the braid assembly including interstitial spaces between filars, filar arrays and the like, is infiltrated by the outer sleeve.

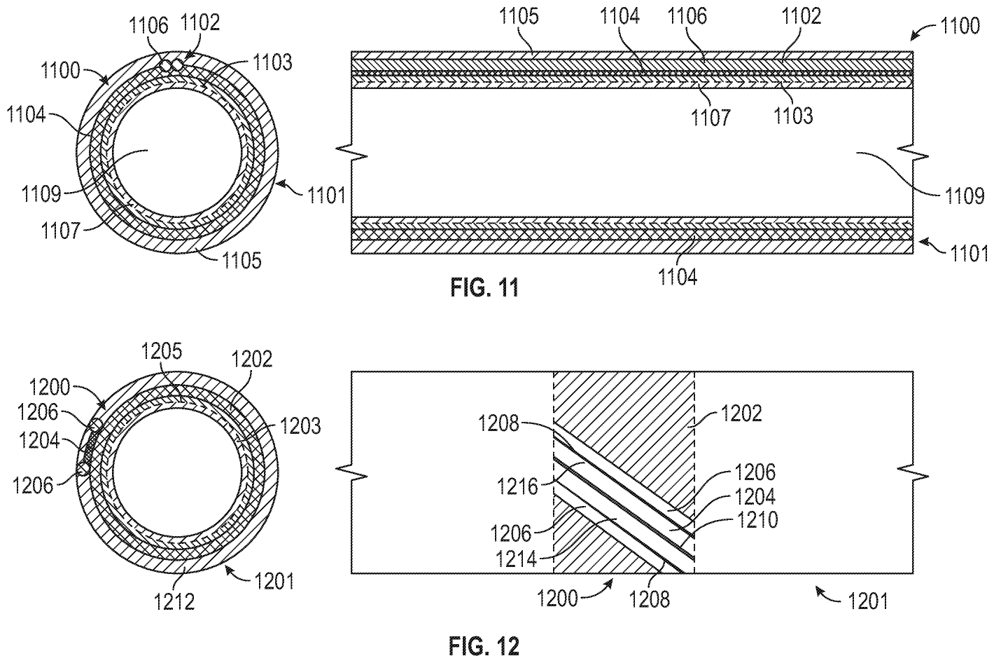

The braid assembly is constructed with a multi-filar (e.g., 6-30 filars) helically wound interlaced braid structure. In some embodiments, the filars are swaged, such that one or more of the filars is partially flat or ovular (e.g., including rectangular and elliptical) in cross-section to achieve a tight wire matrix. In other examples, the braid assembly is made with one or more non-swaged, round, square or rectangular filars (optionally in combination with other filars having the swaged configuration). In one example, the one or more filars one or both of the first and second filar arrays include filars approximating the dimensions and characteristics of a coil (e.g., a circular or ovular cross section, material characteristics such as Young's modulus, flexural modulus or the like). One example of a braided brace 1102 is shown in FIG. 11 by the circular (coil) filars as part of the braid assembly 1100 (and shown in the cross-sectional view on the right taken along the longitudinal axis of the catheter).

In various embodiments, the wall thickness of the braid assembly 1100 ranges from about 0.0005 to 0.020 inches thick. The braid assembly 1100 improves the mechanical characteristics of the catheter 1101, such as compared to current guide catheters with respect to kinking, buckling, flexibility, radial strength, and maintaining circularity of the catheter lumen 1109 cross-section. The braid assembly 1100 of the catheter also improves characteristics of the catheter including, but not limited to, one or more torqueability, flexibility, pushability or kink resistance. This improvement is achieved in one example by varying number of filars (e.g., filars, coils or the like) in each of the filar arrays 1104, 1106 (e.g., in various ratios including but not limited to, 15:1, 14:2, 13:3, 12:4, 11:5, 10:6, 9:7, 8:8, 7:9, 6:10, 5:11, 4:12, 3:13, 2:14, 1:15 with a total filar count of 16 filars). One example of a braid assembly 1200 including an unbalanced ratio is shown in FIG. 12 that includes a 16 filar count example braid having 14 first filars in the first filar array 1202 and 2 second filars in the second filar array 1204. For illustration purposes interlacing is removed (but present in the braid assembly).

In another example, the one or more improved mechanical characteristics are achieved by varying dimensions (e.g., dimensions in cross section) of one or more of the filars in one or more of the arrays. For instance, in arrays with the ratio 14:2 the first filars (14 filars of a first filar array) include first filar dimensions in the cross section such as one or more of diameter, thickness or width less than corresponding dimensions of the second filars (2 filars of a second filar array). Stated another way, the second filars are fewer in number and are larger in at least one cross sectional dimension relative to the first filars. The second filars structurally support the more narrow first filars in the manner of a braided brace, and thereby provide a frame, skeleton, cage or the like that maintains the first filars in a desired configuration (e.g., without kinking or buckling during deflection). One example of a braid assembly 1200 including filars having different dimensions between the first and second filar arrays 1202, 1204 is shown in FIG. 12. As shown the second filars (e.g., coils, filars or the like) of the second filar array 1204 have at least one larger dimension relative to the first filars of the first filar array 1202. In one example, the second filars have a dimension, such as width, at least one order of magnitude larger than the first filars.

Optionally, the second filars include one or more filars, such as coils, interlaced with the first filars. An example of second filars including coils (including filars having coil shapes and dimensions) is provided in FIG. 11. Although a single array 1106 of second filars is shown in FIG. 11, other examples include multiple arrays of the second filars interlaced with a corresponding number of first filar arrays 1104. The one or more interlaced coils are another example of a braided brace for the first filars. The first filars (and the second filars including the braided brace 1102) provide improved torqueability to the catheter, while the braided brace 1102 structurally supports the braid assembly 1100 and provides enhanced kink resistance (and optionally other improved characteristics including pushability and torqueability). The inclusion of a braided brace 1102 incorporates the profile of the brace into the braid assembly and thereby minimizes the inclusion of additional support structures (such as coils) over or beneath the braid assembly with attendant consumption of space (or enlargement of the catheter) avoided.

In still another example, the catheters described herein include a coil 1206 wound along the braid assembly 1200, for instance in a guide recess 1208 provided by one of the filar arrays 1202, 1204. One example of discrete coils is shown in FIG. 12 by the one or more coils 1206 extending along the second filar array 1204. The coil 1206 enhances the mechanical characteristics of the catheter 1201. Optionally, the coil 1206 extends helically along one or more of the filars including for example the braided brace 1210. The coil 1206 and the braided brace 1210 cooperate to capture and hold the first filars (described above) 1202 in place within the catheter 1201. Further, infiltration of the outer sleeve 1212 (e.g., a reflowed or shrunk sleeve) into the braid assembly 1200 and the optional coils 1206 fixes each of the braided brace 1210 and the coil 1206 (optional) in place. The outer sleeve 1212 and the braided brace 1210 capture and hold the first filars 1202 in place and minimize (e.g., eliminate or decrease) kinking of the catheter 1201. Where the coil 1206 is included along the braid assembly 1200, the coil and the braided brace are captured within the outer sleeve 1212 (e.g., reflowed) and clamp the first filars 1202 therebetween. Kinking, buckling or the like of the first filars is thereby resisted by one or more of the braided brace 1210 or the coil 1206 in combination with the outer sleeve 1212.

As further shown in FIG. 12, the catheter 1201 further includes one or more guide recesses 1208 adjacent to the second filar array 1204. The one or more faces 1214, 1216 of the second filar array 1204 form the guide recesses 1208. For instance, as shown in FIG. 12, the second filar array 1204 includes proximal and distal faces 1214, 1216. The one or more guide recesses 1208 follow the helical track of one of the filar arrays 1202, 1204 (in the example shown the second array 1204) and optionally include two guide recesses, one along the proximal face 1214 and the other along the distal face 1216 of the second filar array 1204. Because the second filar array 1204 is interlaced with the first filar array 1202 to form the braid assembly 1200 proximal and distal faces 1214, 1216 are present where the second filar array 1204 is on the exterior of the braid assembly (e.g., between passes of the first filars of the first filar array 1202). In at least those zones, the coil (or coils) 1206 is partially received within the braid assembly 1200 to thereby minimize the space (e.g., outer sleeve 1212 thickness) used to contain the coils 1206 in the outer sleeve. In one example, the braid assembly 1200 facilitates the inclusion of a discrete coil 1206 and the benefits to the mechanical characteristics (e.g., kink-resistance or the like) while minimizing the space needed to retain the coil 1206 in the catheter 1201. At least a portion of the profile of the coils 1206 is concealed within the guide recesses 1208 while the remainder is within the outer layer 1212. The outer layer 1212 is in one example made thinner with its exterior immediately adjacent to the outer surface of the (recessed) coils.

As previously described herein one or more of the ratio of filars, dimensions of filars and components included with the braid assembly 1100, 1200 and along the braid assembly are varied to provide specified mechanical characteristics for a catheter 1101, 1201. For instance, various ratios and dimensions of filars are used to provide a specified torqueability for the catheter 1101, 1201 and at the same time enhance kink-resistance. One or more optional discrete coils 1206 are optionally provided along the braid assembly 1100, 1200, for instance within guide recesses 1208 of the braid assembly 1200, to further enhance the mechanical characteristics of the catheter. Tables 1, 2 and 3 provided herein describe each of the various features of an example braid assembly including, but not limited to, filar ratios between first and second filar arrays, filar dimensions, filar shapes, discrete coils and positioning of the same. These features are chosen and implemented in the catheter to provide the specified characteristics for a therapeutic or diagnostic procedure.

Table 1 (below) provides one example of a braid assembly including a filar count of 16 total filars. As shown, the ratios between the first and second filar arrays, and in some examples their cross sectional shapes, are varied between each of the example braid configurations. Although the smaller filar arrays (e.g., six and under) include the option for circular filars (e.g., coils or the like), circular filars are also included in some examples with filar array having larger filar counts, for instance greater than six filars.

TABLE 1
Structural Braid Configuration (example of 16 filar
count between arrays, but total filar count is
higher or lower in examples such as 6, 8, 10,
12, 14, 18, 20, 22, 24, 26, 28, 30 or the like)

| | First Filar | | Second Filar | |
|---|---|---|---|---|
| | Array - Filar | | Array - Filar | |
| Braid | Count (e.g., | First Filar | Count (e.g., | Second Filar |
| Config- | left hand | Array - Filar | right hand | Array - |
| uration | helix) | Shape | helix) | Filar Shape |
| X1 | 15 | flat or ovular | 1 | circular |
| | | | | (e.g., coil), |
| | | | | flat or ovular |
| X2 | 14 | flat or ovular | 2 | circular |
| | | | | (e.g., coil), |
| | | | | flat or ovular |
| X3 | 13 | flat or ovular | 3 | circular |
| | | | | (e.g., coil), |
| | | | | flat or ovular |
| X4 | 12 | flat or ovular | 4 | circular |
| | | | | (e.g., coil), |
| | | | | flat or ovular |
| X5 | 11 | flat or ovular | 5 | circular |
| | | | | (e.g., coil), |
| | | | | flat or ovular |
| X6 | 10 | flat or ovular | 6 | circular |
| | | | | (e.g., coil), |
| | | | | flat or ovular |
| X7 | 9 | flat or ovular | 7 | flat or ovular |
| X8 | 8 | flat or ovular | 8 | flat or ovular |
| X9 | 7 | flat or ovular | 9 | flat or ovular |
| X10 | 6 | circular | 10 | flat or ovular |
| | | (e.g., coil), | | |
| | | flat or ovular | | |
| X11 | 5 | circular | 11 | flat or ovular |
| | | (e.g., coil), | | |
| | | flat or ovular | | |
| X12 | 4 | circular | 12 | flat or ovular |
| | | (e.g., coil), | | |
| | | flat or ovular | | |
| X13 | 3 | circular | 13 | flat or ovular |
| | | (e.g., coil), | | |
| | | flat or ovular | | |
| X14 | 2 | circular | 14 | flat or ovular |
| | | (e.g., coil), | | |
| | | flat or ovular | | |
| X15 | 1 | circular | 15 | flat or ovular |
| | | (e.g., coil), | | |
| | | flat or ovular | | |

Table 2 describes filar configurations including dimensions for each of the first and second filar arrays. In some examples, larger filar dimensions (e.g., of the second filar array) are paired with smaller filar dimensions of the other filar array (e.g., the first filar array). Examples of filars having circular cross sections are also provided including dimensions between about 0.001 to 0.01 inches. Table 2 further qualitatively shows the relative difference between dimensions of the first and second filar arrays in the Order of Magnitude column. As shown, at least some the filar configurations provide one or more of the filars of one array as at least one order of magnitude larger in a dimension, such as width or diameter, relative to the corresponding dimension of the other filars of the other (e.g., second) filar array.

TABLE 2
Filar Configurations (example flat/ovular or coil dimensions used
in some examples with the braid configuration of Table 1)

| | | | | | Order of |
|---|---|---|---|---|---|
| | First | | Second | | Magnitude |
| | Filar | First Filar | Filar | Second Filar | Difference |
| Filar | Array - | Array - Filar | Array - | Array - Filar | (e.g., of |
| Config- | Filar | Dimensions | Filar | Dimensions | width or |
| uration | Shape | (inches) | Shape | (inches) | diameter) |
| Y1 | flat or | 0.0005 × 0.003 | flat or | 0.002 × 0.015 | Yes |
| | ovular | | ovular | | |
| Y2 | flat or | 0.0005 × 0.003 | flat or | 0.002 × 0.010 | Yes |
| | ovular | | ovular | | |
| Y3 | flat or | 0.002 × 0.015 | flat or | 0.002 × 0.015 | No |
| | ovular | | ovular | | |
| Y4 | flat or | 0.002 × 0.015 | circular | 0.001 | No |
| | ovular | | (e.g., coil) | | |
| Y5 | flat or | 0.005 × 0.015 | circular | 0.003 | No |
| | ovular | | (e.g., coil) | | |
| Y6 | flat or | 0.0005 × 0.003 | circular | 0.004 | Yes |
| | ovular | | (e.g., coil) | | |
| Y7 | flat or | 0.0005 × 0.003 | circular | 0.005 | Yes |
| | ovular | | (e.g., coil) | | |
| Y8 | flat or | 0.0005 × 0.003 | circular | 0.008 | Yes |
| | ovular | | (e.g., coil) | | |
| Y9 | flat or | 0.0005 × 0.003 | circular | 0.01 | Yes (2 |
| | ovular | | (e.g., coil) | | orders of |
| | | | | | magnitude) |

Table 2 provides an array of example dimensions.
Filar Dimensions (flat or ovular) vary between thicknesses of 0.0005 to
0.005 and widths of 0.001 to 0.030
As described herein, in one example, a filar array with fewer filars has
relatively larger filar dimensions relative to a companion filar array
having a greater number of filars

Table 3 describes the placement of one or more discrete coils with the braid assembly. Further, the table describes options for positioning of the coils within guide recesses, for instance along the one or more guide recesses 1208 previously shown in FIG. 12 and provided alongside one of the filar arrays, for instance the second filar array 1204 having larger dimensions and correspondingly larger recesses (e.g., having a depth corresponding to the thickness or diameter of the second filars). In various examples, one or more coils are positioned within proximal or distal guide recesses 1208 (e.g., along the proximal or distal faces 1214, 1216 of a filar assembly such as the second filar assembly).

TABLE 3
Discrete Coil (example coils optionally
used in some examples with the braid
configurations of Tables 1 and 2)

| | Placement | Coil | Proximal or | |
|---|---|---|---|---|
| | of Coil | Positioning | Distal Guide | |
| | Relative | in Braid | Recess (Relative | |
| Coil | to Braid | Guide | to braid filar or | Number |
| Config- | (Exterior | Recess or | filars providing | of |
| uration | or Interior) | Recesses | guide recesses) | Coils |
| Z1 | Exterior | Yes | Proximal | 1 |
| Z2 | Exterior | Yes | Proximal and | 2 |
| | | | Distal | |
| Z3 | Exterior | Yes | Distal | 1 |
| Z4 | Exterior | No | NA | 1 |
| Z5 | Exterior | No | NA | 2 |
| Z6 | Interior | No | NA | 1 |
| Z7 | Interior | No | NA | 2 |

Coil Dimensions: Various including 0.001 to 0.010 inches.

A variety of prophetic example catheter configurations are provided herein. The configurations are drawn by assembling one or more of the configurations provided in Tables 1, 2 and 3 and provide variation in mechanical characteristics based on the configurations chosen (e.g., the inclusion of a braided brace, such as larger filars in one of the arrays, a coil or the like improve kink-resistance).

The catheter of example 1 includes a braid assembly provided over a PTFE inner liner with an intermediate tie layer provided between the PTFE and the braid assembly (e.g., to facilitate coupling of the braid assembly and optionally an outer sleeve with the inner liner). The inner liner has an outer diameter of about 0.255 to 0.256 inches; the tie layer outer diameter (over the inner liner) is about 0.260 to 0.2605 inches; and the braid assembly outer diameter (over the tie layer) is about 0.270 to 0.271 inches. An outer sleeve, such as Pebax or the like, is provided along the catheter and over the braid assembly.

The catheter of example 2 includes a braid assembly with a discrete coil extending along the braid assembly exterior. The braid assembly includes an 8:8 ratio of first filars to second filars. In one example, the first filars have cross sectional dimensions of about 0.002 (thickness) by about 0.015 (width) inches. The 25 second filars have the same dimensions. In another example, the second filars have the previously described dimensions (e.g., 0.002×0.015 inches) while the first filars have cross sectional dimensions of about 0.0005 inches by 0.003 inches. In this example, the second filars have dimensions an order of magnitude greater than the first filars. The braid assembly is provided over a PTFE inner liner with an intermediate tie layer. The inner liner has an outer diameter of about 0.2555 to 0.2565 inches; the tie layer outer diameter (over the inner liner) is about 0.2585 to 0.259 inches; and the braid assembly outer diameter (over the tie layer) is about 0.261 to 0.2615 inches. The coil is loaded over the braid assembly and retained therealong with the outer sleeve, such as Pebax or the like. In one example, the outer sleeve has a durometer of 55D and an outer diameter of between about 0.280 inches to 0.285 inches. Optionally, an end of the coil is fixed near the corresponding end of the braid assembly and the coil is wound around the braid assembly in the same direction (e.g., from proximal to distal). In another example, the coil is would along one or more guide recesses of one of the filar arrays as described herein.

The catheter of example 3 includes a braid assembly having an 8:8 ratio of eight filars for the first filar array and eight filars for the second filar array. The braid assembly is provided over a PTFE inner liner and an intermediate tie layer is provided between the PTFE and the braid assembly (e.g., to facilitate coupling of the braid assembly and optionally an outer sleeve with the inner liner). The inner liner has an outer diameter of about 0.255 to 0.266 inches; the tie layer outer diameter is about 0.258 to 0.259 inches; and the braid assembly outer diameter is about 0.263 to 0.264 inches. The first filars have cross sectional dimensions of 0.0005 (thickness) by 0.003 (width) inches. The second filars have cross section dimensions of 0.002 (thickness) by 0.015 (width) inches. The second filars have dimensions an order of magnitude greater than the first filars. The braid assembly is braided at 50 picks per inch, and is encapsulated with a polymer having a durometer of 55D and an outer diameter of about 0.280 to 0.285 inches.

In other examples (related to example 3), the braid assembly has a 14:2 or 12:4 ratio with the first filars having the same dimensions. In this example, the picks per inch are optionally increased (e.g., greater than 50, for instance to 180 PPI). In still another example, the braid assembly has a 15:1 ratio of first filars to second filars. Each of the first and second filars include the same dimensions as those for the 8:8 example provided immediately above.

In either of the examples (e.g., ratios of 8:8, 14:2, 12:4, 15:1) the catheter optionally includes a discrete coil. For instance, in the last example including the 15:1 ratio the coil is positioned within a guide recess formed along the second filar array (e.g., including the single second filar). The guide recess appears in the examples as one or more rifled grooves extending along the braid assembly. In addition to providing increased kink-resistance, the inclusion of the coil enhances the radial strength of the catheter (resistance to collapsing).

The catheter of example 4 includes a braid assembly having a 14:2 ratio of fourteen first filars for the first filar array and two second filars for the second filar array. In a similar manner to the previous examples, the braid assembly is provided over a PTFE inner liner (e.g., inner sleeve 1107 in FIG. 11 and inner sleeve 1203 in FIG. 12) and an intermediate tie layer (e.g., 1103, 1205, respectively) is provided between the PTFE and the braid assembly. The inner liner has an outer diameter of about 0.255 to 0.256 inches; the tie layer outer diameter is about 0.258 to 0.259 inches; and the braid assembly outer diameter is about 0.263 to 0.2635 inches. The first filars have cross sectional dimensions of 0.0005 (thickness) by 0.003 (width) inches. The second filars have cross section dimensions of 0.002 (thickness) by 0.015 (width) inches and are an order of magnitude greater than the first filars. Optionally, the two filars of the second filar array are staggered 180 degrees apart (e.g., on opposite sides of the catheter) to form a double helix. The braid assembly is braided at 90 PPI, and is encapsulated with a polymer (e.g., outer sleeve 1105) having a durometer of 55D and an outer diameter of about 0.280 to 0.285 inches.

The catheter of example 5 includes a braid assembly having a 15:1 ratio of fifteen first filars for the first filar array and one second filar for the second filar array. The filars are provided at 160 PPI. In a similar manner to the previous examples, the braid assembly is provided over a PTFE inner liner and an intermediate tie layer is provided between the PTFE and the braid assembly. The inner liner has an outer diameter of about 0.2555 to 0.256 inches; the tie layer outer diameter is about 0.258 to 0.259 inches; and the braid assembly outer diameter is about 0.2625 to 0.2635 inches. The first filars have cross sectional dimensions of 0.0005 (thickness) by 0.003 (width) inches. The second filar has cross sectional dimensions of 0.002 (thickness) by 0.015 (width) inches and is an order of magnitude greater than the first filars.

The second filar array in this example provides at least one guide recess (e.g., along the proximal or distal faces of the filar array) and a coil is wound along the filar array and positioned within the guide recess (the recess serves as a guide for placement of the coil). The coil is at least partially received within the guide recess and the profile of the coil is thereby decreased because it is partially absorbed by the filar array and its guide recess. In one example, where the coil is wound in a particular direction (e.g., left hand) the second filar array is also wound left handed (and the first filar array wound right handed) to ensure placement of the coil within the guide recess.

The braid assembly and the coil are encapsulated with a polymer (outer sleeve) having a durometer of 55D and an outer diameter of about 0.280 to 0.285 inches. Optionally, the outer sleeve is reflowed multiple time (e.g., at least twice) to remove gas bubbles in the sleeve. The catheter of example 5 has enhanced radial strength and flexibility relative to at least some of the other examples.

Example 6 includes a selection of catheters including braid assemblies having ratios of 15:1, 14:2, 12:4 and so on. The braid assemblies include second filars having dimensions approaching or equaling those of a coil. For instance, filar (coil) diameters of about 0.003 to 0.005 inches (e.g., larger than the 0.002×0.015 filars described herein). The second filars of the second filar array are interlaced with the first filar array. In one example, the second filars are staggered around the catheter body, for instance according to the count of the second filars (4 second filars at 90 degree intervals, 3 at 120 degree intervals, 2 at 180 degree intervals or the like). Optionally, these catheters and the second filar arrays of each are paired with discrete coils that are positioned within one or more guide recesses of the second filar array.

FIG. 13 shows one example of a catheter assembly 100 having a hub 108 coupled with the catheter shaft 102 (e.g., a shaft, for instance a main tubular shaft, or the like). As further shown in FIG. 13, a graduated strain relief fitting 1300 is interposed between at least a portion of the hub 108 and the catheter shaft 102. As described herein, the graduated strain relief fitting 1300 is included in one or more catheters optionally having other features including (but not limited to) one or more of the braid assembly 112, braid assembly 608, braid assembly 808, braid assembly 814, braid assembly 1100, braid assembly 1200, strain relief fitting 110, braid assembly 2700, or the like.

As further shown in FIG. 13, the graduated strain relief fitting 1300 includes a fitting body 1302 constructed with one or more of polymers, metal, composites or the like. The graduated strain relief fitting 1300 (e.g., the fitting body 1302) extends from a fitting proximal portion 1304 to a fitting distal portion 1306. The fitting proximal portion 1304 is coupled along with the hub 108 to interface with the hub 108 and accordingly minimize (e.g., lower, reduce or eliminate) stress risers therebetween. The fitting distal portion 1306 provides a fitting interface between the graduated strain relief fitting 1300 and the catheter shaft 102. For instance, in one example, the fitting distal portion 1306 and a corresponding portion of the catheter shaft 102, such as a shaft proximal portion 106, include the fitting interface. The fitting distal portion 1306 of the graduated strain relief fitting 1300, as described herein, is configured to minimize (e.g., lower, reduce or eliminate) stress risers otherwise incident between the catheter shaft 102 and the interface with the graduated strain relief fitting 1300 during navigation, deflection or manipulation of the catheter assembly 100

Referring again to FIG. 13, the catheter shaft 102 extends from the shaft proximal portion 106 to the shaft distal portion 104. As shown in FIG. 13, the catheter shaft 102, in this example, has a shaft profile 1310 smaller than a corresponding hub profile 1308 of the hub 108. As further described herein, the graduated strain relief fitting 1300 minimizes stress risers, for instance, between the hub 108 and the catheter shaft 102. In one example, the gradated strain relief fitting 1300 includes a tapered profile configured to transition the catheter assembly 100 from the hub 108 (and the larger hub profile 1308) to the shaft 102 (and the smaller shaft profile 1310).

Additionally, and as described herein, the graduated strain relief fitting 1300 minimizes (e.g., lowers, decreases or eliminates) stress risers at the interfaces between the fitting proximal portion 1304 and the hub 108 (a hub interface) as well as at the fitting distal portion 1306 and the catheter shaft 102 (a fitting interface). The graduated strain relief fitting 1300 includes one or more flexure joints 1312 configured to modulate (e.g., control, tune, graduate or the like) one or more support characteristics, such as flexural modulus, of the graduated strain relief fitting 1300 to provide a specified flexibility to the graduated strain relief fitting 1300 that maintains the support provided to the catheter shaft 102 at the interfaces with the fitting distal portion 1306 as well as the interface with the hub 108, for instance, at the fitting proximal portion 1304. Accordingly, the graduated strain relief fitting 1300, including the flexure joints 1312, provides a specified modulated flexural modulus at each of these interfaces and along the graduated strain relief fitting 1300 to control the one or more support characteristics (e.g., flexural modulus, elastic modulus, tensile modulus or the like) to minimize kinking, buckling or the like of the catheter shaft 102, for instance, when deflected.

In the view shown in FIG. 13, the one or more flexure joints 1312 include a helical groove, scallop or the like extending along the graduated strain relief fitting 1300. In the example shown the one or more flexure joints 1312 extend between the fitting proximal portion 1304 and the fitting distal portion 1306. As described herein, the one or more flexure joints 1312 include, but are not limited to, grooves, scallops, scoring, flutes, notches, recesses, dimples or the like provided at one or more locations along the graduated strain relief fitting 1300. For instance, the flexure joints 1312 are provided at one or more locations or pitches (e.g., frequencies, joints per unit length or the like) to provide enhanced flexibility to the graduated strain relief fitting 1300 at a specified location or locations. In another example, the flexure joints 1312 are provided at one or more profiles, for instance, having a same or similar shape but one or more larger or smaller sizes to accordingly modulate the flexural modulus of the graduated strain relief fitting 1300 at specified locations. In still other examples, the profiles of the flexure joints 1312 include a variety of profiles (e.g., shapes, sizes, depth relative to the fitting surface, combinations of the same or the like) that modulate the flexural modulus of the fitting 1300 at one or more specified locations.

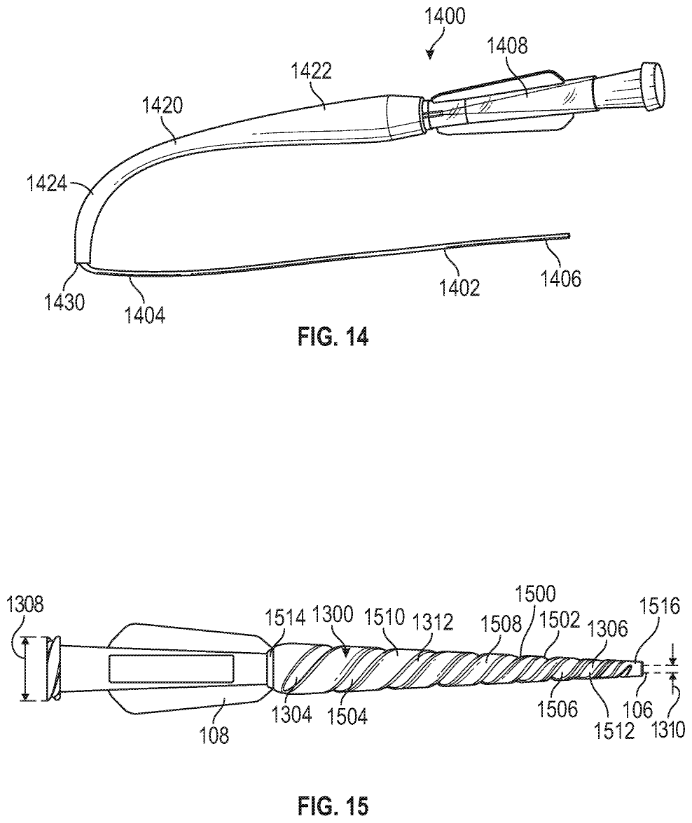

FIG. 14 shows another catheter assembly 1400. In this example, the catheter assembly 1400 includes a hub 1408 coupled with a catheter shaft 1402. A strain relief fitting 1420 is interposed between the hub 1408 and the catheter shaft 1402. The strain relief fitting 1420 includes a tapered configuration extending from a fitting proximal portion 222 to a fitting distal portion 1424. As shown, the shaft proximal portion 1404 extends to a shaft distal portion 1406. In one example, the shaft proximal portion 1404 extends within the strain relief fitting 1420 and is coupled with the hub 1408.

The catheter assembly 1400 in FIG. 14 is in a deflected configuration, for instance, with one or more of lateral deflections, twisting or the like of the catheter shaft 1402 relative to the hub 1408. A stress riser 1430 is included at the interface between the fitting distal portion 1424 and the shaft proximal portion 1404. In the example shown the stress riser 1430 kinks the catheter shaft 1402 at the interface between the fitting distal portion 1424 and the shaft proximal portion 1404. In some examples, the support characteristic of the strain relief fitting 1420 at the fitting distal portion 1424 provides a robust strain relief fitting 1420 that supports the catheter shaft 1402 proximal to the fitting distal portion 1424. The remainder of the catheter shaft 1402 extending to the shaft distal portion 1406 is without this support. Accordingly, with deflection of the catheter shaft 1402 a stress riser 1430 is imparted to the catheter shaft 1402 at the interface of the fitting distal portion and the shaft proximal portion 1404. The stress 1430 causes kinking at the interface between the fitting distal portion 1424 and the shaft proximal portion 1404. In one example, the flexural modulus of the fitting distal portion 1424 is greater than the corresponding flexural modulus of the catheter shaft 1402. Accordingly, when the catheter assembly 1400 is deflected the fitting distal portion 1424 deflects to a limited degree while the catheter shaft 1402 bends, kinks or the like in a more pronounced fashion relative to the fitting distal portion.

FIG. 15 shows a detailed side view of the hub 108 and the graduated strain relief fitting 1300 previously shown in FIG. 13. As shown, the graduated strain relief fitting 1300 extends from the hub 108, for instance, from a hub interface 1514 proximate the hub 108 to a fitting interface 1516 proximate the shaft proximal portion 106 extending from the fitting 1300. The shaft proximal portion 106 extends within the strain relief fitting 1300 to the hub 108. In one example, the catheter shaft is received and coupled with the hub 108 within an interior orifice of the hub 108. The graduated strain relief fitting 1300 provides the hub interface 1514 and the fitting interface 1516.

As shown in FIG. 15, the graduated strain relief fitting 1300 includes one or more flexure joints 1312 provided along the fitting 1300. For instance, a strain relief profile 1500 of the flexure joints 1312 includes, but is not limited to, one or more of grooves, scallops, scoring, flutes, notches, recesses, dimples or the like along the graduated strain relief fitting 1300. In this example, the strain relief profile 1500 extends from the fitting proximal portion 1304 to the fitting distal portion 1306. In another example, the strain relief profile 1500 is located at one or more specified locations of the fitting 1300, for instance proximate to the fitting distal portion 1306, one or more other locations of the fitting or the like.

In one example, the strain relief profile 1500 is a consistent profile extending between the fitting proximal and distal portions 1304, 1306. In another example, the strain relief profile 1500 changes from the fitting proximal portion 1304 to the fitting distal portion 1306. For instance, one or more of the shape, size, frequency or the like of the flexure joint 1312 changes between the proximal and distal portions 1304, 1306. In the example shown in FIG. 15, a first joint pitch 1504, for instance, the number of flexure joints 1312 per unit length is less than a second joint pitch 1506 proximate to the fitting distal portion 1306. The increase in joint pitch (e.g., the second joint pitch 1506 in this example) decreases the wall thickness of the graduated strain relief fitting 1300 at the fitting distal portion 1306 and the fitting interface 1516. The increase in joint pitch modulates the support characteristic of the fitting 1300 at the fitting interface 1516, for instance to a value similar to the support characteristic (e.g., flexural modulus) of the shaft proximal portion 106. In this example, the flexural modulus of the fitting distal portion 1306 more closely approximates the flexural modulus of the shaft proximal portion 106. As described herein, by controlling a support characteristic such as flexural modulus at the fitting distal portion 1306 the fitting distal portion 1306 readily deflects with the shaft proximal portion 106 while at the same time also supporting the shaft proximal portion 106 under deflection. Accordingly, one or more of kinking, buckling, underlying stress risers or the like are minimized at the fitting interface 1516 between the fitting distal portion 1306 and the shaft proximal portion 106.

In another example, the graduated strain relief fitting 1300 includes a fitting frame 1508 between the one or more flexure joints 1312. The fitting frame 1508 is, in one example, a portion of the graduated strain relief fitting 1300 having a wall thickness greater than the corresponding wall thickness proximate to the flexure joints 1312. Accordingly, the fitting frame 1508 provides enhanced support to one or more portions of the graduated strain relief fitting 1300 while the flexure joints 1312 modulate the support provided by the graduated strain relief fitting 1300. Changes in one or more of the frequency of the fitting frame 1508, frequency of the flexure joints 1312 or the like are used in various examples to provide one or more specified support characteristics, such as flexural modulus, at one or more locations of the graduated strain relief fitting 1300 to correspondingly support the catheter shaft 102 during deflection while at the same time minimizing stress risers, kinking, buckling or the like.

In the example shown in FIG. 15, the fitting frame 1508 includes a first frame pitch 1510 proximate to the fitting proximal portion 1304 that is greater relative to a second frame pitch 1512 associated with the fitting distal portion 1306. With the greater first frame pitch 1510 (e.g., frequency, area or length of the frame per unit length or the like) proximate to the fitting proximal portion 1304, the support provided by the graduated strain relief fitting 1300 is accordingly enhanced relative to the lesser second frame pitch 1512, for instance, proximate to the fitting distal portion 1306.

In another example, the graduated strain relief fitting 1300 includes a taper between the fitting proximal and distal portions 1304, 1306. As shown in FIG. 15, the fitting 1300 tapers toward the distal portion 1306. The taper of the graduated strain relief fitting 1300 is another example of a feature configured to modulate the support characteristics of the fitting, for instance to provide support to the catheter shaft 102 and at the same minimize stress risers, kinking, buckling or the like. For example, the wall thickness of the graduated strain relief fitting (including the fitting body 1302, FIG. 13) is gradually decreased from the fitting proximal and distal portions 1304, 1306 to according permit enhanced deflection proximate to the distal portion 1304 while supporting the catheter shaft 102. In still another example, the taper cooperates with the features described herein including one or more of flexure joints 1312, fitting frame 1508, variations in the same or the like to modulate one or more support characteristics of the fitting 1300 at one or more locations (e.g., along the catheter shaft 102, proximate to the hub 108 or the like).

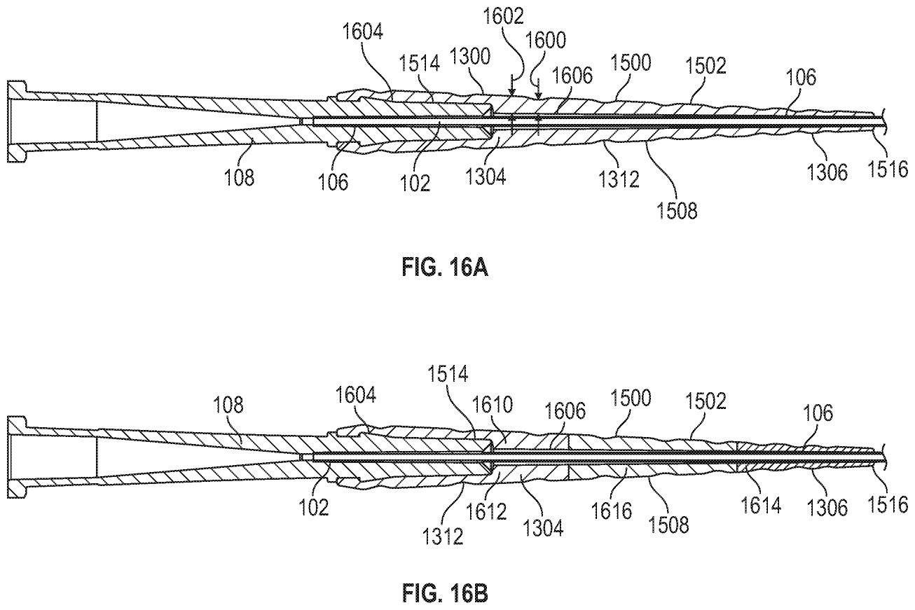

FIG. 16A shows a cross-sectional view of an example graduated strain relief fitting 1300. In this example, the hub 108 is a varied configuration or profile relative to the hub 108 previously shown in FIG. 15. As shown in FIG. 16A a portion of the hub 108 is received within a hub socket 1604 of the graduated strain relief fitting 1300. Similarly, the catheter shaft 102 is received within a shaft channel 1606 of the graduated strain relief fitting 1300. As further shown in FIG. 16A a portion of the shaft proximal portion 106 is received within a corresponding portion of the hub 108. The hub 108 optionally includes a channel, port or the like configured to receive the shaft proximal portion 106 therein. Accordingly, in this example, the catheter shaft 102 extends through the fitting distal portion 1306 along the shaft channel 1606 through the fitting proximal portion 1304 and into the hub 108.

As further shown in FIG. 16A, the graduated strain relief fitting 1300 includes the strain relief profile 1500 having one or more of the flexure joints 1312 and intervening portions of the graduated strain relief fitting 1300, for instance, corresponding to the fitting frame 1508 shown in FIG. 15. As shown in FIG. 16A, a first wall thickness 1600 corresponding, for instance, to the wall thickness provided proximate to the flexure joints 1312 is less than a corresponding second wall thickness 1602 provided between the flexure joints 1312 in the fitting frame 1508. The second wall thickness 1602, in one example, corresponds to the wall thickness of the fitting frame 1508 between the flexure joints 1312. As further shown in FIG. 16A, the second wall thickness 1602 and the associated fitting frame 1508 assume a larger proportion of the overall strain relief profile 1500 proximate to the fitting proximal portion 1304. Conversely, the first wall thickness 1600 and the associated flexure joints 1312 proximate to the fitting distal portion 1306 assume a larger portion of the strain relief profile 1500. As previously described with regard to FIG. 15, the variation in the fitting frame 1508 as well as the flexure joints 1312 including, for instance, various changes in profiles of the flexure joints 1312 or fitting frame 1508, frequencies (e.g., pitch) or the like modulates the support provided by the graduated strain relief fitting 1300 in a specified manner.

In the example shown in FIG. 16A, by providing additional flexure joints 1312, a higher frequency of flexure joints (second joint pitch 1506 in FIG. 15) and a corresponding lower frequency of the fitting frame (second frame pitch 1512) proximate to the fitting distal portion 1306, the support characteristic provided by the fitting distal portion 1306 is decreased, for instance, to correspond with or closely approximate one or more mechanical characteristics of the shaft proximal portion 106 including, for instance, a flexural modulus of the catheter shaft 102. Conversely, the ratio of the fitting frame 1508 to the flexure joints 1312 including, for instance, a first frame pitch 1510 and a first joint pitch 1504 (as shown in FIG. 15) is modulated at the fitting proximal portion 1304 to accordingly bolster or enhance the support characteristics of the graduated strain relief fitting 1300 proximate to the hub interface 1514, and thereby minimize a sharp decrease of the support characteristic at the hub interface 1514 relative to the robust material of the hub 108. Accordingly, by modulating the support provided at the hub interface 1514 and the fitting interface 1516, the support characteristics of the graduated strain relief fitting 1300 are modulated or tuned to correspond to the structural specifications of the shaft proximal portion 106 at a plurality of locations. The graduated strain relief fitting thereby minimizes kinking, buckling, stress risers or the like while at the same time providing support at each of the hub interface 1514 and the fitting interface 1516.

FIG. 16B is another cross-sectional view of a graduated strain relief fitting 1610 of a catheter assembly. The graduated strain relief fitting 1610, shown in FIG. 16B, is similar in at least some regards to the graduated strain relief fitting 1300 shown in FIG. 16A. For instance, the fitting 1610 includes a hub socket 1604 configured for reception of a portion of a hub 808 therein. A shaft channel 1606 extends through the strain relief fitting 1610 and is concentric with a corresponding orifice within the hub 108. Additionally, the catheter shaft 102 extends through the graduated strain relief fitting 1610 along the shaft channel 1606 and is received in the hub socket 1604. As further shown in FIG. 16B, the graduated strain relief fitting 1610 includes a strain relief profile 1500 including one or more flexure joints 1312, a fitting frame 1508 or the like between the fitting proximal portion 1304 and the fitting distal portion 1306. As previously described, the fitting frame 1508 and flexure joints 1312 cooperate with the material of the graduated strain relief fitting 1610 to modulate (e.g., control, tune, modify or the like) the support characteristics of the graduated strain relief fitting 1610, for instance, at one or more of the hub interface 1514 and the fitting interface 1516.

As further shown in this example, the graduated strain relief fitting 1610 includes one or more fitting materials, such as a first fitting material 1612 and a second fitting material 1614. The first fitting material 1612 includes a higher support characteristic (e.g., flexural modulus, tensile modulus, rigidity or the like) relative to the second fitting material 1614 associated with a fitting distal portion 1306. Accordingly, the second fitting material 1614 provides a more flexible distal portion 1306 to correspond and flexibly support the shaft proximal portion 106 at the fitting interface 1516. Conversely, the first fitting material 1612 associated with the fitting proximal portion 1304 has a higher support characteristic (e.g., flexural modulus, tensile modulus, rigidity or the like) than the fitting distal portion 1306. Accordingly, additional support is provided at the hub interface 1514 to the catheter shaft 102 to maintain the catheter shaft 102 in a relatively linear configuration relative to the hub 108 at the hub interface 1514.

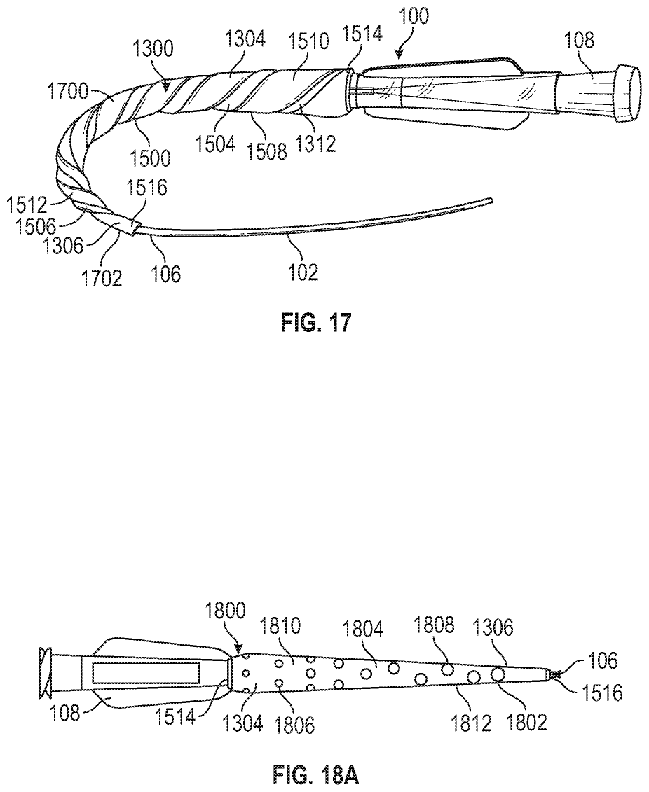

In another example, the graduated strain relief fitting 1610 includes one or more supplemental materials including, for instance, a third fitting material 416, for instance, interposed between the first and second fitting materials 1612, 1614. In one example, the third fitting material 416 is a more flexible material than that used in the first fitting material 1612 and a less flexible material than the second fitting material 1614. For instance, the third fitting material 416 provides an intermediate support characteristic (e.g., flexural modulus or the like) relative to the flexural moduli of the fitting proximal and distal portions 1304, 1306. In still other examples, the fitting materials 1612, 1614, 416 include the same material, and the material is selectively doped or treated to provide differing support characteristics. For instance, the first fitting material 1612 includes a fitting filler including, but not limited to, metallic particles, glass fibers or the like configured to enhanced the support characteristic of the first fitting material 1612 and the associated fitting proximal portion 1304. In this example, the second fitting material 1614 includes a lesser amount of the fitting filler (including no filler) to provide a flexible fitting distal portion 1306 to conform or provide a complementary profile of the graduated strain relief fitting 1610 to the catheter shaft (see FIG. 17). In another example, the fitting filler includes one or more materials, reticulations, pores or the like that decrease the support characteristic and enhance the flexibility. In this permutation additional fitting filler is provided with the second fitting material 1614 to enhance flexibility while still providing support, and a lesser amount of the fitting filler is provided in the first fitting material 1612 to enhance support while decreasing flexibility.

FIG. 17 shows the catheter assembly 100 including the graduated strain relief fitting 1300. Optionally, the catheter assembly 100, shown in FIG. 17, is used with the graduated strain relief fitting 1600 shown in FIG. 16B (including, for instance, one or more of variations in material, profile, both or the like). Referring again to FIG. 17, the catheter assembly 100 is shown in a deflected configuration 1700 including one or more of bending or twisting of the catheter shaft 102. As shown, the graduated strain relief fitting 1300 assumes a complementary profile 1702 to the deflected catheter shaft 102 in contrast to the configuration shown in FIG. 14 including the stress riser 1430 and corresponding kink in the catheter shaft 1402.

As previously described, the graduated strain relief fitting 1300 includes one or more of flexure joints 1312, a fitting frame 1508, variations in material or the like. The fitting 1300, including one or more of these features, is configured to provide one or more supporting characteristics to the catheter shaft 102, for instance at the hub interface 1514 between the graduated strain relief fitting 1300 and the hub 108, and the fitting interface 1516 between the fitting distal portion 1306 of the strain relief fitting 1300 and the shaft proximal portion 106. As shown in FIG. 17, the shaft proximal portion 106 is in a deflected curved configuration. In contrast to FIG. 14, the catheter assembly 100, shown in FIG. 17, includes the catheter shaft 102 in the deflected configuration 1700 without kinks, buckling or the like. Instead, the strain relief fitting 1300 provides a complementary profile 1702 to the deflected catheter shaft 102.

The fitting distal portion 1306 includes a support characteristic configured to provide flexibility in the fitting distal portion 1306 while at the same time supporting the shaft proximal portion 106 in the deflected configuration 1700. For example, the flexure joints 1312, joint pitch, fitting frame 1508, frame pitch, materials or the like are configured to provide a specified support characteristic at the fitting distal portion 1306 including the fitting interface 1516. The support characteristic (e.g., a second flexural modulus) is modulated proximate to the fitting distal portion 1306 relative to a first higher flexural modulus of the fitting proximal portion 1304 with one or more of variations in the flexure joints 1312, fitting frame 1508, their respective pitches, materials of the fitting or the like. The modulated support characteristic of the fitting distal portion 1306 permits deflection of the catheter shaft 102 and the fitting distal portion 1306 into a configuration and respective complementary profile 1702 like that shown in FIG. 17. At the same time the graduated strain relief fitting 1300 having the complementary profile 1702 also supports the catheter shaft 102 while deflected to minimize (e.g., decreasing, eliminating or the like) events that complicate procedures, such as kinking or buckling of the catheter shaft 102.

Conversely, the fitting proximal portion 1304 including, for instance, one or more of a thicker wall, an increased frame pitch 1510 relative to the frame pitch 1512, decreased joint pitch 1504 relative to the joint pitch 1506, variations in material or the like provides enhanced support to the catheter shaft 102 proximate to the hub interface 1514. Accordingly, the catheter shaft 102 remains in a substantially linear configuration relative to the hub 108 even in the deflected configuration 1700. In one example, the support characteristic of the fitting proximal portion 1304, such as a first flexural modulus, is greater than the second flexural modulus at the fitting distal portion 1306. Optionally, the flexural modulus of the proximal portion 1304 approaches a corresponding flexural modulus of the hub 108.

The graduated strain relief fitting 1300, including one or more of the flexure joints 1312 (e.g., grooves, scallops, scoring, flutes, notches, recesses, dimples or the like), the fitting frame 1508, modulation of their respective profiles or pitch, as well as variations in material are used separately or together to modulate the support characteristics of the graduated strain relief fitting 1300 to accordingly provide enhanced support characteristics at one or more locations while permitting deflection of the catheter shaft (and optionally assuming the complementary profile 1702).

In one example, the second flexural modulus of the fitting distal portion 1306 includes a flexural modulus less than or equal to the flexural modulus of the catheter shaft 102 to support the catheter shaft and assume the complementary profile 1702. In another example, the second flexural modulus of the fitting distal portion 1306 approaches the flexural modulus of the catheter shaft 102. For instance, the second flexural modulus substantially matches the modulus of the catheter shaft (e.g., is equal to or within 1 to 5 percent above or below the modulus, within 1,000, 7,000, 10,000 psi or the like). In another example, the fitting proximal portion 1304 includes a first flexural modulus greater than the flexural modulus of the fitting distal portion 1306 and accordingly greater than the flexural modulus of the catheter shaft 102. The first flexural modulus optionally approaches the modulus of the hub 108 (e.g., is equal to or within 20 to 25 percent of the hub modulus, within 10,000, 20,000 or 50,000 psi or the like). In one example, with these modulated support characteristics the graduated strain relief fitting 1300 is configured to assume the complementary profile 1702 in the deflected configuration 1700 and support the catheter shaft 102 while at the same time minimizing one or more of kinking, buckling or the like. Optionally, flexural modulus as used herein is used interchangeably with similar mechanical characteristics, such as modulus of elasticity (Young's modulus), tensile strength or the like.

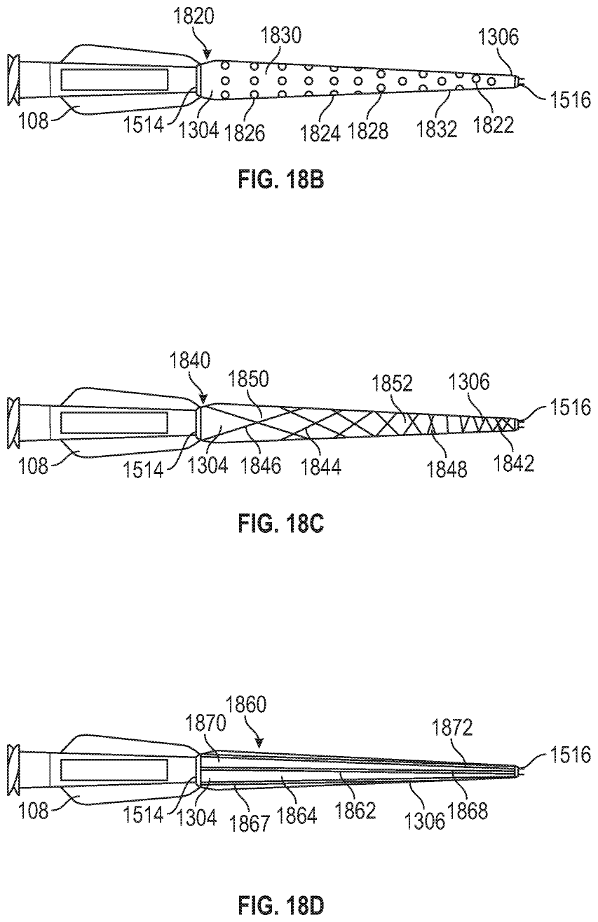

FIGS. 18A-D show examples of graduated strain relief fittings 1800, 1820, 1840, 1860. In each of these examples, the strain relief fittings include one or more flexure joints, fitting frames, pitches (e.g., frequency of the flexure joints, fitting frame or the like) to illustrate example variations of these features useable with the graduated strain relief fittings described herein. Referring first to FIG. 18A, the example graduated strain relief fitting 1800 includes one or more features similar to the previously described strain relief fitting, such as the fitting 1300. For instance, the strain relief fitting 1800 is coupled with a hub 108 at a hub interface 1514 and is coupled with a shaft proximal portion 106 of the catheter shaft. As further shown in FIG. 18A, a fitting interface 1516 is between the fitting distal portion 1306 of the graduated strain relief fitting 1800 and the shaft proximal portion 106. Conversely, the hub interface 1514 is between the fitting proximal portion 1304 and the hub 108.

Referring again to FIG. 18A, the graduated strain relief fitting 1800 includes one or more flexure joints 1802 between the fitting proximal and distal portions 1304, 1306. In this example, the flexure joints 1802 include scallops, recesses, dimples or the like provided along the graduated strain relief fitting 1800. As shown in FIG. 18A, the flexure joints 1802 have an increasing profile (in this example, size), from the fitting proximal portion 1304 to the fitting distal portion 1306. For instance, the flexure joints 1802 provided proximate to the fitting proximal portion 1304 are smaller than those proximate to the fitting distal portion 1306. Conversely, the fitting frame 1804 is between the flexure joints 1802 from the fitting proximal portion 1304 to the fitting distal portion 1306. In this example, the fitting frame 1804 increases, for instance, has a greater surface area (per unit length) as the graduated strain relief fitting 1800 extends from the fitting distal portion 1306 to the fitting proximal portion 1304.

As shown in FIG. 18A, the relationship between the flexure joints 1802 (scallops, recesses, dimples or the like) including profile, frequency or the like is shown with pitches, including first and second joint pitches 1806, 1808. In this example, the first joint pitch 1806 is less than the second joint pitch 1808. For instance, the flexure joints 1802 have a decreased profile (e.g., one or more of cross-section, shape, size, dimensions, contour, radius, perimeter, circumference, diameter, outline, boundary, configuration, pattern, arrangement, thickness, or the like). proximate to the fitting proximal portion 1304 relative to the flexure joints 1802 proximate to the fitting distal portion 1306. In another example, the flexure joints 1802 have a gradually increasing profile (and corresponding pitch) from the fitting proximal portion 1304 to the fitting distal portion 1306. Accordingly, the first and second joint pitches 1806, 1808 are, in one example, a continuum of joint pitches that gradually increase from the fitting proximal portion to the fitting distal portion 1304, 1306.

As further shown in FIG. 18A, in this example, first and second frame pitches 1810, 1812 of the fitting frame 1804, conversely decrease between the fitting proximal portion 1304 and the fitting distal portion 1306. As shown in FIG. 18A, the frame pitches include intervening space between flexure joints corresponding to portions of the fitting 1300 having an increased wall thickness relative to proximate flexure joints 1802. The first frame pitch 1810 proximate to the fitting proximal portion 1304 is larger than the second frame pitch 1812 proximate the fitting distal portion 1306. Accordingly, as the frame pitch decreases toward the fitting distal portion 1306 (and the joint pitch increases to the fit 1808) the graduated strain relief fitting 1800 provides enhanced flexibility proximate to the fitting distal portion.

Conversely, the graduated strain relief fitting 1800 provides enhanced support proximate to the fitting proximal portion 1304 according to the first frame pitch 1810 (and counterpart first joint pitch 1806) relative to the second frame pitch 1812 (and counterpart second joint pitch 1808) at the fitting distal portion 1306. Accordingly, the fitting distal portion 1306 provides a support characteristic for the shaft proximal portion 106 configured to facilitate bending of the shaft proximal portion 106 but at the same time support the shaft proximal portion 106 and minimize (e.g., decrease, eliminate or the like), kinking, buckling or the like of the shaft. One example of the deformation is shown in the deflected configuration 1700 in FIG. 17 including the complementary profile 1702.

Optionally, the fitting distal portion 1306, including variations in the flexure joints 1802 and fitting frame 1804, provide a support characteristic, such as flexural modulus, approximating the flexural modulus of the catheter shaft 102. In another example, the flexural modulus of the fitting distal portion 1306 of the graduated strain relief fitting 1800 includes a flexural modulus less than or equal to the flexural modulus of the catheter shaft 102. The flexural modulus of the fitting distal portion 1306, when approximating the catheter shaft 102 (equal to or less than, within 1 to 5 percent of the catheter shaft or the like) facilitates the supported deformation of the catheter shaft 102 while the fitting 1800 assumes a supporting complementary profile, such as the profile 1702, shown in FIG. 17.

FIG. 18B shows another example of a graduated strain relief fitting 1820. In this example, the flexure joints 1822 of the strain relief fitting 1820 have a consistent profile while providing another example of a variation in pitch. For instance, the scallops, recesses, dimples or the like provided along the graduated strain relief fitting 1820 have a consistent shape and size. In contrast, the joint pitch of the flexure joints 1822 (e.g., frequency, number of flexure joints per unit length or the like) increases from the fitting proximal portion 1304 to the fitting distal portion 1306 of the fitting 1820. Accordingly, with additional flexure joints 1822 proximate to the fitting distal portion 1306, the profile of the graduated strain relief fitting 1820 has an enhanced recessed configuration proximate to the fitting distal portion 1306. Conversely, the fitting frame 1824 interposed between the flexure joint 1822 in this example has an increased surface area (an example of frame pitch) proximate to the fitting proximal portion 1304.

As further shown in FIG. 18B, the first joint pitch 1826 with the consistent profile flexure joints 1822 is less than the second joint pitch 1828 proximate the fitting distal portion 1306. Conversely, the first frame pitch 1830 proximate to the fitting proximal portion 1304 is greater in comparison to the second frame pitch 1832 proximate to the fitting distal portion 1306. As shown in FIG. 18B, the relationship between the various pitches is optionally similar to the pitches shown in FIG. 18A. For example, the frame pitch gradually decreases from the proximal to the distal portions 1306, while the joint pitch gradually increases. Accordingly, the fitting distal portion 1306 provides support to the shaft proximal portion 106 while at the same time flexibly deforming with deflection of the shaft proximal portion 106, for instance, into a complementary configuration, such as the configuration 1702 shown in FIG. 17.

FIG. 18C shows another example of a graduated strain relief fitting 1840. In this example, the flexure joints 1842 include, but are not limited to, one or more grooves, scallops, scoring, flutes, notches or the like provided along the graduated strain relief fitting 1840. For instance, the flexure joints 1842 include one or more grooves or scoring provided at angles, orientations or the like along the graduated strain relief fitting 1840 to modulate the support characteristics of the strain relief fitting 1840 between the fitting proximal and distal portions 1304, 1306. For instance, as shown in FIG. 18C, the first joint pitch 1846 and the second joint pitch 1848 of the fitting proximal and distal portions 1304, 1306 vary. In FIG. 18C the angles of the flexure joints (another example of joint pitch) decrease or approach a similar orientation to the longitudinal axis of the graduated strain relief fitting 1840 as the flexure joints 1842 progress toward the fitting proximal portion 1304. Conversely, the flexure joints 1842 have a greater pitch (e.g., angle, orientation relative to the longitudinal axis or the like) as the joints approach the fitting distal portion 1306. Conversely, the fitting frame 1844, in this example between the flexure joints 1842 includes a greater first frame pitch 1850 (e.g., area per unit length) proximate to the fitting proximal portion 1304 relative to a second frame pitch 1852 proximate to the fitting distal portion 1306.

FIG. 18D shows another example of a graduated strain relief fitting 1860. In this example, the strain relief fitting 1860 includes flexure joints 1862 (gaps, grooves, scallops, recesses or the like) extending in a longitudinal fashion, for instance, along the strain relief fitting between the fitting proximal portion 1304 and fitting distal portion 1306. As shown in FIG. 18D, the flexure joints 1862 have an angled configuration optionally corresponding to the taper of the graduated strain relief fitting 1860. Accordingly, the first joint pitch 1867 (in this example, ratio of joint area to frame area or the like) proximate to the fitting proximal portion 1304 is less than the second joint pitch 1868 proximate to the fitting distal portion 1306. As shown in FIG. 18D, the flexure joints 1862 are packed, clustered or the like proximate to the fitting distal portion 1306. In contrast the flexure joints 1862 proximate to the fitting proximal portion 1306 are relatively spaced apart, and accordingly include the lesser first joint pitch 1867.

Conversely, the pitch of the fitting frame 1864 interposed between the flexure joints 1862 decreases between the fitting distal and proximal portions 1306, 1304. Accordingly, the first frame pitch 1870 (e.g., frame area per unit length, ratio of frame area to the joint area or the like) proximate to the fitting proximal portion 1306 is greater than the second frame pitch 1872 proximate to the fitting distal portion 1306. In a similar manner to the other graduated strain relief fittings described herein, the flexure joints 1862 and the fitting frame 1864 cooperate to modulate the support characteristics of the graduated strain relief fitting 1860, for instance, at the fitting interface 1516 and the hub interface 1514.

As shown in FIG. 18D, the fitting frame 1864 having the higher first frame pitch 1870 proximate to the fitting proximal portion 1304 supports the hub interface 1514 with the increased wall thickness of the frame relative to the joints 1862. Conversely, the flexure joints 1862 having a higher second joint pitch 1868 (e.g., clustering, density or the like) proximate the fitting distal portion 1306 enhance the flexibility of the graduated strain relief fitting 1860 while, at the same time, providing support to the shaft proximal portion 106. As previously described, the modulation (tuning, varying, controlling or the like) of the support characteristics of the fitting, such as flexural moduli, facilitates the supported deformation of the catheter shaft such as the catheter shaft 102, shown in FIG. 17, into the example deflected configuration 1700 including the example complementary profile 1702 shown in FIG. 17. Stated another way, the shaft proximal portion 106 is readily deflected into the configuration 1700 shown in FIG. 17 and at the same time is supported by the strain relief fitting 1860 (as well as the other examples described herein) in a complementary profile.

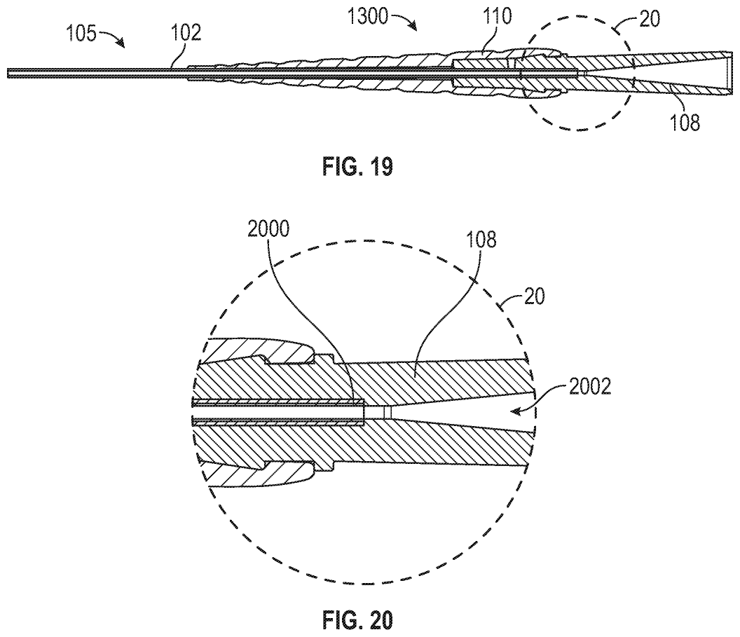

FIG. 19 is a cross-sectional view of the catheter 100 of FIG. 1 at the line 19-19. As described herein, in an example the catheter 100 includes a catheter body 102, and the proximal portion 105 of the catheter body 102 is coupled with the hub 108. In some examples, the catheter assembly 100 includes the strain relief fitting 110. For instance, catheter assembly 100 shown in FIG. 19 includes the graduated strain relief fitting 1300 (also shown in FIG. 13) coupled along the catheter body 102.

FIG. 20 is a detailed cross-sectional view of the catheter assembly 100 of FIG. 19 at the circle detail 20. The catheter body 102 includes a shaft lumen 2000, and the shaft lumen 2000 optionally receives one or more instruments, for instance a guidewire, catheter, diagnostic or therapeutic instrument or the like. In an example, the shaft lumen 2000 is in communication with a hub lumen 2002 of the hub 108. The hub lumen 2002 optionally tapers toward the shaft lumen 2000, for instance to facilitate insertion of instruments into the shaft lumen 2000.

FIG. 21 is a side view of the catheter body 102. As described herein, the catheter body 102 extends between the proximal portion 105 and the distal portion 103 having the distal tip 104. In an example, the distal tip 104 includes a coil 2100, and the coil 2100 has varying imaging characteristics to facilitate identification of the distal tip 104 (e.g., including one or more of location, orientation or the like) relative to other portions of the catheter assembly 100. For instance, the varying imaging characteristics of the distal tip 104 facilitate identification of the distal tip 104 relative to the proximal portion 105 of the catheter shaft 120.

FIG. 22 is a detailed side view of the catheter body 102 of FIG. 21 at the box detail 22 in FIG. 21. As described herein, the catheter body 102 includes the outer coating 610. FIG. 22 shows portions of the outer coating 610 partially hidden for clarity to expose other aspects of the catheter assembly 100, including the coil 2100.

The distal portion 103 of the catheter assembly 100 has varying imaging characteristics to facilitate identification of the distal portion 103 relative to other portions of the catheter assembly 100, for example identification of the distal portion 103 relative to the proximal portion 105 or intermediate portions of the catheter. In an example, a first section 2200 of the coil 2100 has a first imaging characteristic 2202 (e.g., radiopacity, radiodensity, radiopaqueness, radiolucentness, ultrasound opacity or the like). The coil 2100 includes a second section 2204, and the second section 2204 of the coil 2100 has a second imaging characteristic 2206. In an example, the first imaging characteristic 2022 differs from the second imaging characteristic 2206, for instance to provide contrast to the distal tip 104 relative to the remainder of the catheter body 102 (e.g., the proximal portion 103 of the catheter body 102). In some examples, the variation of characteristics, profiles of the sections or the like facilitate identification of the catheter including the location of the distal portion 103, its orientation or the like. In an example, the difference between the first imaging characteristic 2202 and the second imaging characteristic 2206 emphasizes visibility (e.g., observation, classification, discernment, distinguishing, determination of orientation or the like) of the distal tip 104 relative to the remainder of the catheter body 102. In another example, the difference between the first imaging characteristic 2202 and the second imaging characteristic 2206 contrasts the first section 2200 of the coil 2100 from the second section 2204 of the coil 2100. For instance, the first section 2200 is radiopaque based on the first imaging characteristic 2202, and the second section 2204 is radiolucent based on the second imaging characteristic 2206.

In yet another example, the difference between the first imaging characteristic 2202 and the second imaging characteristic 2206 emphasizes visibility of the first section 2200 of the coil 2100 as distinct from the second section 2204 of the coil 2100. Accordingly, the difference between the imaging characteristic 2202 of the first section 2200, and the imaging characteristic 2206 of the section 2204 facilitates identification of the distal tip 104 relative to other portions of the catheter body 102, such as the proximal portion 105 (shown in FIG. 21). Thus, in some examples, the catheter assembly 100 does not include a marker (e.g., a radiographic marker, such as a band included in the distal tip 104, or the like), and instead includes one or more sections 2200, 2204 that facilitate identification of a portion of the catheter, such as the distal portion 103.

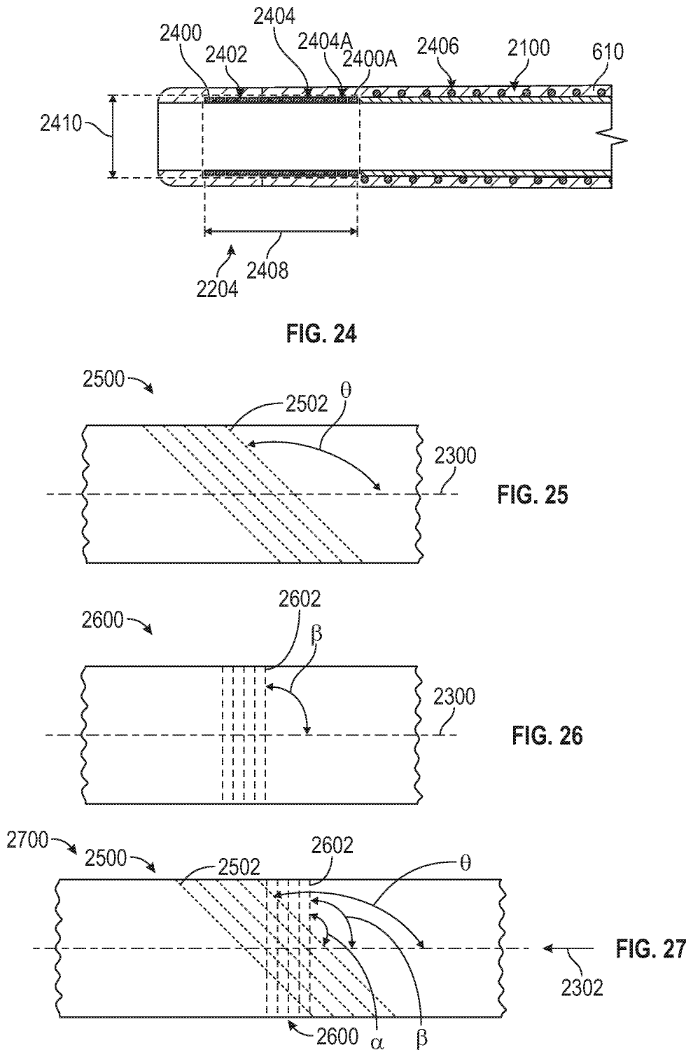

FIG. 23 is a cross-sectional view of the of the catheter body 102 of FIG. 22 at the line 23-23. As described herein, in some examples, the second section 2204 of the coil 2100 is swaged. In an example, the second section 2204 is swaged according to one or more operations, including (but not limited to) grinding, etching, planarizing, stamping, crimping, mechanical removal (or the like) of a portion of the second section 2204 to swage the second section 2204 of the coil 2100. As shown in detail in FIG. 24 the swaged second section 2204 has a planar profile (in comparison to the first section 2200).

Referring to FIG. 23, the catheter assembly 100 includes a longitudinal axis 2300 extending along the catheter body 102. In an example, an axial force 2302 is optionally applied to the catheter, for example along the longitudinal axis 2300, manipulate the catheter body 102 through vasculature. In an example, the axial force 2302 applies compression along the catheter body. In some approaches, compression of the catheter assembly 100 or manipulation including deflection or the like increases the risk of buckling or kinking the catheter body 102. As described herein, the coil 2100 enhances the strength of the catheter body 102, enhances the performance of the catheter assembly 100, and conversely minimizes the risk of kinking or buckling.

FIG. 24 is a detailed cross-sectional view of the catheter body 102 of FIG. 23 at the box detail 24. As described herein, the second section 2204 of the coil is swaged. For instance, the distal tip 104 includes one or more filars 2400. The one or more filars 2400 optionally include a planar filar profile 2402. In an example, the second section 2204 of the coil 2100 has the planar filar profile 2402. In another example, the planar filar profile 2402 includes a plurality of component planar perimeter surfaces 2404 that form the profile. For instance, a first filar 2400A of the one or more filars 2400 has a first planar perimeter surface 2404A. In an example, a coating is coupled along the plurality of planar perimeter surfaces 2404. For instance, the outer layer 610 is in an example a coating coupled along the plurality of planar perimeter surfaces 2404. In yet another example, the first section 2200 of the coil 2100 has a curved planar perimeter surface 2406. FIG. 24 shows the outer layer 610 coupled along the curved planar perimeter surface 2406 of the first section 2200 of the coil 2100.

In an example, the second section 2204 of the coil 2100 has a first characteristic 2408 (e.g., length, width, radius, diameter, perimeter, area or the like). In another example, the second section 2204 of the coil 2100 has a second characteristic 2410 (e.g., length, width, radius, diameter, perimeter, area or the like). In one example, the first characteristic 2408 is in this example a length within a range of approximately 0.02 inches to approximately 0.07 inches (however the present subject matter is not so limited). In yet another example, the second characteristic 2410 is in this example a length within a range of approximately 0.015 inches to approximately 0.025 inches (however the present subject matter is not so limited).

FIG. 25 is a schematic view of another example of the catheter 100 including a first braided layer 2500. As described herein, the catheter body includes one or more filars, such as a strand fiber, filament, or the like. In an example, the one or more filars are included in a braided layer, such as the first braided layer 2500. As described herein, the first braided layer 2500 cooperates with other components of the catheter assembly 100 to minimize failure of the catheter shaft 102, for instance through kinking, buckling or the like. In an example, the first braided layer 2500 has a first braid profile (shown in short dashed lines in FIG. 25) having an angle (e.g., is transverse or misaligned) with respect to the longitudinal axis 2300. For example, the first braided layer 2500 includes a first filar 2502 with first angle θ from the longitudinal axis 2300.

FIG. 26 is a schematic view of another example of the catheter 100 including a second braided layer 2600. The first and second braided layers 2500, 2600 are, in an example, component layers of a braid assembly including interleaved or interwoven filars of the layers 2500, 2600. In one example, the second braided layer 2600 has a second braid profile (shown in long dashed lines FIG. 26), and the second braid profile extends at an angle with respect to the longitudinal axis 2300. For example, a second filar 2602 of the second braided layer 2600 is at a second angle β with respect to the longitudinal axis 2300. In an example, the second angle β is different than the first angle θ (shown in FIG. 25) of the first filar 2502 of the first braid layer 2500. Accordingly, in some examples the first braided layer 2500 having the first braid profile (shown in FIG. 25 with short dashed lines) is oriented at a different angle relative to the second braided layer 2600 having the second braid profile (shown in FIG. 26 with long dashed lines).

FIG. 27 is a schematic view of the catheter 100 having a braid assembly 2700 including the first braided layer 2500 and the second braided layer 2600. FIG. 27 shows the first braided layer 2500 having the first braid profile (shown with short dashed lines) rotationally offset with respect to the second braided layer 2600 having the second braid profile (shown with long dashed lines). In an example, the angle between the braided layers 2500, 2600 facilitates varying rates of expansion between the braided layers 2500, 2600. In an example, expansion includes (but is not limited to) one or more of volumetric expansion, circumferential expansion, radial expansion, cross-sectional area expansion, or the like. For instance, the first braided layer 2500 expands at a first rate when loaded in compression (e.g., when an axial pushing force is applied to the catheter 100). The second braided layer 2600 surrounds the first braided layer 2500 and expands at a second different rate (e.g., lesser rate). The expansion of the second braided layer 2600 at the second (lesser) rate constrains and braces the first braided layer 2500 as described herein, for instance when the force 2302 is applied to the catheter assembly 100.

In an example, the expansion of the second braided layer 2600 at the second rate is less than the expansion of the first braided layer 2500 at the first rate. For instance, the first rate of expansion for the first braided layer 2500 is greater than the second rate of expansion of the second braided layer 2600 because of the angle of the first filar 2502 of the first braided layer 2500. Conversely, the angle of the second filar 2602 is less than the angle of the first filar 2502 and the second filar 2602 and the associate second braided layer 2600 more slowly expand with axial loading of the catheter 100. In one example, the second braided layer 2600 surrounds and engages with the first braided layer 2500, and thereby braces the first braided layer 2500 in the manner of a belt, girdle or ring. Accordingly, the second braided layer 2600 constrains and braces the first braided layer 2500, for instance due to the difference in rate of expansion between the first braided layers 2500 and the second braided layer 2600 when axially loaded. For instance, the constraint or bracing of the first braided layer 2500 by the second braided layer 2600 minimizes the risk of one or more of buckling, kinking or the like of the catheter body 102 during axial loading of the catheter assembly 100 (e.g., with compression, deflection or the like).

Referring to FIG. 27 and in an example, the first braided layer 2500 has the first braid profile (indicated with short dashed lines) and the second braided layer 2600 has the second braid profile (indicated with long dashed lines). The first braid profile is at an angle from the longitudinal axis 2300 by the angle θ, and the second braid profile is at an angle from the longitudinal axis by the angle β. Accordingly, in this example, the first braid profile is at an angle relative to the second braid profile by the angle α. In an example, the angle θ is approximately 45 degrees. The angle β is approximately 90 degrees. Thus, the first braid profile (shown in short dashed lines) is at angle of approximately 45 degrees with respect to the second braid profile (shown in long dashed lines). In an example, the angle (e.g., at the angle α) between the first braid profile and second braid profile facilitates the expansion of the first braid layer 2500 at the first rate while the second braided layer 2600 expands at the second (lesser) rate. Accordingly, the second braided layer 2600 constrains and braces the first braided layer 2500, for instance to minimize one or more of kinking, buckling or the like of the catheter body 102.

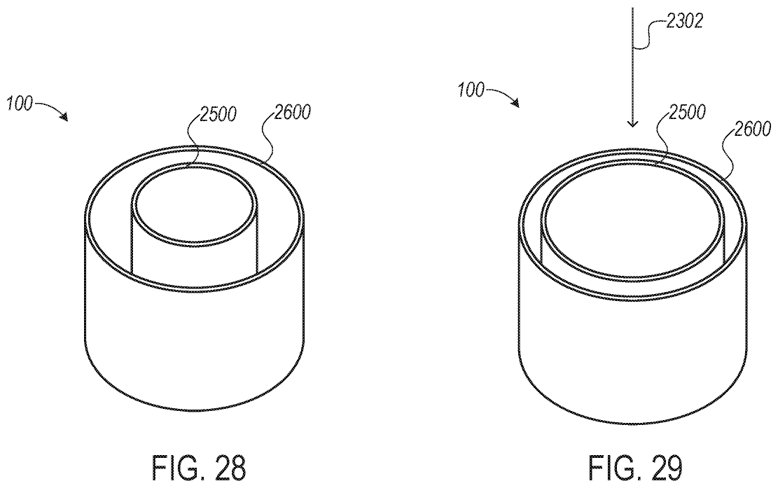

FIGS. 28 and 29 are schematic views of the first braided layer 2500 and the second braided layer 2600 in an initial configuration and an expanded configuration (respectively). The variation in expansion rate of the first braided layer and the second braided layer 2600 is exaggerated in FIGS. 28 and 29 relative to the actual variation. In an example, the expansion of the second braided layer 2600 at the second rate is less than the expansion of the first braided layer 2500 at the first rate. For instance, the first rate of expansion for the first braided layer 2500 is greater than the second rate of expansion of the second braided layer 2600 because of the angle of the first filar 2502 of the first braided layer 2500. In one example, the second braided layer 2600 surrounds and engages with the first braided layer 2500, and thereby braces the first braided layer 2500 in the manner of a belt, girdle or ring. Accordingly, the second braided layer 2600 constrains and braces the first braided layer 2500, for instance due to the difference in rate of expansion between the first braided layers 2500 and the second braided layer 2600 when axially loaded, such as with axial force 2302.

### Various Notes & Examples

Example 1 is a catheter assembly, comprising: a catheter body extending from a catheter proximal portion to a catheter distal portion having a distal tip; a coil proximate to the catheter distal portion, the coil having a first section and a second section, wherein: the first section of the coil extends toward the distal tip, and the first section has a first imaging characteristic; the second section is swaged and extends from the first section to the distal tip, and the second section of the coil has a second imaging characteristic; and the second imaging characteristic differs from the first imaging characteristic, and the different first and second imaging characteristics are configured to contrast and emphasize visibility of the distal tip relative to the remainder of the catheter body.

In Example 2, the subject matter of Example 1 optionally includes wherein the second section of the coil includes a planar filar profile, and the planar filar profile includes a plurality of planar perimeter surfaces of swaged filars included in the swaged second section of the coil.

In Example 3, the subject matter of any one or more of Examples 1-2 optionally include a tip coating applied along a planar filar profile at the distal tip, and the planar filar profile includes a plurality of planar perimeter surfaces of swaged filars included in the swaged second section.

In Example 4, the subject matter of any one or more of Examples 1-3 optionally include wherein the coil includes platinum.

In Example 5, the subject matter of any one or more of Examples 1-4 optionally include wherein the catheter does not include a marker coupled to the catheter body proximate to the distal tip.

In Example 6, the subject matter of any one or more of Examples 1-5 optionally include inches.

In Example 7, the subject matter of any one or more of Examples 1-6 optionally include inches.

Example 8 is a catheter assembly, comprising: a catheter body extending from a catheter proximal portion to a catheter distal portion; a first braided layer within the catheter body; a second braided layer coupled along an exterior of the first braided layer; wherein the first and second braided layer include first and second, respective, filar arrays; and wherein the first and second braided layers are configured to expand to an expanded configuration with the application of axial force along a catheter longitudinal axis, and in the expanded configuration: the first braided layer is configured to expand at a first rate; the second braided layer is configured to expand at a second rate less than the first rate of expansion, and the expansion at the second rate is configured to constrain and brace the first braided layer.

In Example 9, the subject matter of Example 8 optionally includes wherein the first braided layer has a first braid profile, the second braided layer has a second braid profile, and the second braid profile is at an angle with respect to the first braid profile.

In Example 10, the subject matter of Example 9 optionally wherein the first braid profile is at an angle of approximately 45 degrees relative to a longitudinal axis of the catheter, and the second braid profile is at an angle of approximately 90 degrees relative to the longitudinal axis.

In Example 11, the subject matter of any one or more of Examples 8-10 optionally include a coil proximate to the catheter distal portion, the coil having a first section and a second section, wherein: the first section of the coil extends toward the distal tip, and the first section has a first imaging characteristic; the second section is swaged and extends from the first section to the distal tip, and the second section of the coil has a second imaging characteristic; and the second imaging characteristic differs from the first imaging characteristic, and the different first and second imaging characteristics are configured to contrast and emphasize visibility of the distal tip relative to the remainder of the catheter body.

In Example 12, the subject matter of any one or more of Examples 8-11 optionally include wherein the second braided layer constrains and braces the first braided layer and minimizes kinking of the catheter body.

Example 13 is a catheter assembly, comprising: a catheter body extending from a catheter proximal portion to a catheter distal portion having a distal tip; a coil proximate to the catheter distal portion, the coil having a first section and a second section, wherein: the first section of the coil extends toward the distal tip, and the first section has a first imaging characteristic; the second section is swaged and extends from the first section to the distal tip, and the second section of the coil has a second imaging characteristic; and the second imaging characteristic differs from the first imaging characteristic, and the different first and second imaging characteristics are configured to contrast and emphasize visibility of the distal tip relative to the remainder of the catheter body.

In Example 14, the subject matter of Example 13 optionally includes wherein the second section of the coil includes a planar filar profile, and the planar filar profile includes a plurality of planar perimeter surfaces of swaged filars included in the swaged second section of the coil.

In Example 15, the subject matter of any one or more of Examples 13-14 optionally include a tip coating applied along a planar filar profile at the distal tip, and the planar filar profile includes a plurality of planar perimeter surfaces of swaged filars included in the swaged second section.

In Example 16, the subject matter of any one or more of Examples 13-15 optionally include wherein the coil includes platinum.

In Example 17, the subject matter of any one or more of Examples 13-16 optionally include wherein the catheter does not include a marker coupled to the catheter body proximate to the distal tip.

In Example 18, the subject matter of any one or more of Examples 13-17 optionally include wherein the first braided layer has a first braid profile, the second braided layer has a second braid profile, and the second braid profile is at an angle with respect to the first braid profile.

In Example 19, the subject matter of Example 18 optionally includes wherein the first braid profile is at an angle of approximately 45 degrees relative to a longitudinal axis of the catheter, and the second braid profile is at an angle of approximately 90 degrees relative to the longitudinal axis.

In Example 20, the subject matter of any one or more of Examples 13-19 optionally include wherein the second braided layer constrains and braces the first braided layer and minimizes kinking of the catheter body.

Example 21 may include or use, or may optionally be combined with any portion or combination of any portions of any one or more of Examples 1 through 20 to include or use, subject matter that may include means for performing any one or more of the functions of Examples 1 through 20.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

The claimed invention is:

1. A catheter assembly, comprising:
a catheter body extending from a catheter proximal portion to a catheter distal portion having a distal tip;
a coil proximate to the catheter distal portion, the coil having a first section and a second section, wherein:
the first section of the coil extends toward the distal tip, and the first section has a first imaging characteristic;
the second section is swaged and extends from the first section to the distal tip, and the second section of the coil has a second imaging characteristic; and
the second imaging characteristic differs from the first imaging characteristic, and the different first and second imaging characteristics are configured to contrast and emphasize visibility of the distal tip relative to the remainder of the catheter body.

2. The catheter assembly of claim 1, wherein the second section of the coil includes a planar filar profile, and the planar filar profile includes a plurality of planar perimeter surfaces of swaged filars included in the swaged second section of the coil.

3. The catheter assembly of claim 1 comprising a tip coating applied along a planar filar profile at the distal tip, and the planar filar profile includes a plurality of planar perimeter surfaces of swaged filars included in the swaged second section.

4. The catheter assembly of claim 1, wherein the coil includes platinum.

5. The catheter assembly of claim 1, wherein the catheter does not include a marker coupled to the catheter body proximate to the distal tip.

6. The catheter assembly of claim 1, wherein the second section has a length characteristic within a range of approximately 0.02 inches to approximately 0.07 inches.

7. The catheter assembly of claim 1, wherein the second section has an outer characteristic within a range of approximately 0.015 inches to approximately 0.025 inches.

8. A catheter assembly, comprising:
a catheter body extending from a catheter proximal portion to a catheter distal portion;
a first braided layer within the catheter body;
a second braided layer coupled along an exterior of the first braided layer;
wherein the first and second braided layer include first and second, respective, filar arrays; and
wherein the first and second braided layers are configured to expand to an expanded configuration with the application of axial force along a catheter longitudinal axis, and in the expanded configuration:
the first braided layer is configured to expand at a first rate;
the second braided layer is configured to expand at a second rate less than the first rate of expansion, and the expansion at the second rate is configured to constrain and brace the first braided layer.

9. The catheter assembly of claim 8, wherein the first braided layer has a first braid profile, the second braided layer has a second braid profile, and the second braid profile is at an angle with respect to the first braid profile.

10. The catheter assembly of claim 9, wherein the first braid profile is at an angle of approximately 45 degrees relative to a longitudinal axis of the catheter, and the second braid profile is at an angle of approximately 90 degrees relative to the longitudinal axis.

11. The catheter assembly of claim 8, further comprising:
a coil proximate to the catheter distal portion, the coil having a first section and a second section, wherein:
the first section of the coil extends toward the distal tip, and the first section has a first imaging characteristic;
the second section is swaged and extends from the first section to the distal tip, and the second section of the coil has a second imaging characteristic; and
the second imaging characteristic differs from the first imaging characteristic, and the different first and second imaging characteristics are configured to contrast and emphasize visibility of the distal tip relative to the remainder of the catheter body.

12. The catheter assembly of claim 8, wherein the second braided layer constrains and braces the first braided layer and minimizes kinking of the catheter body.

13. A catheter assembly, comprising:
a catheter body extending from a catheter proximal portion to a catheter distal portion having a distal tip;
a coil proximate to the catheter distal portion, the coil having a first section and a second section, wherein:
the first section of the coil extends toward the distal tip, and the first section has a first imaging characteristic;
the second section is swaged and extends from the first section to the distal tip, and the second section of the coil has a second imaging characteristic; and
the second imaging characteristic differs from the first imaging characteristic, and the different first and second imaging characteristics are configured to contrast and emphasize visibility of the distal tip relative to the remainder of the catheter body.
a first braided layer within the catheter body;
a second braided layer coupled along an exterior of the first braided layer;
wherein the first and second braided layer include first and second, respective, filar arrays; and
wherein the first and second braided layers are configured to expand to an expanded configuration with the application of axial force along a catheter longitudinal axis, and in the expanded configuration:
the first braided layer is configured to expand at a first rate;
the second braided layer is configured to expand at a second rate less than the first rate of expansion, and the expansion at the second rate is configured to constrain and brace the first braided layer.

14. The catheter assembly of claim 13, wherein the second section of the coil includes a planar filar profile, and the planar filar profile includes a plurality of planar perimeter surfaces of swaged filars included in the swaged second section of the coil.

15. The catheter assembly of claim 13 comprising a tip coating applied along a planar filar profile at the distal tip, and the planar filar profile includes a plurality of planar perimeter surfaces of swaged filars included in the swaged second section.

16. The catheter assembly of claim 13, wherein the coil includes platinum.

17. The catheter assembly of claim 13, wherein the catheter does not include a marker coupled to the catheter body proximate to the distal tip.

18. The catheter assembly of claim 13, wherein the first braided layer has a first braid profile, the second braided layer has a second braid profile, and the second braid profile is at an angle with respect to the first braid profile.

19. The catheter assembly of claim 18, wherein the first braid profile is at an angle of approximately 45 degrees relative to a longitudinal axis of the catheter, and the second braid profile is at an angle of approximately 90 degrees relative to the longitudinal axis.

20. The catheter assembly of claim 13, wherein the second braided layer constrains and braces the first braided layer and minimizes kinking of the catheter body.
